(19) United States
(12) Patent Application Publication
(10) Pub. No.: US 2026/0290589 A1
(43) Pub. Date: Sep. 24, 2026

(54) SYSTEM AND METHOD FOR HEALTH ANALYSYS BASED ON PHARMACOGENOMICS AND/OR MICROBIOME

(71) Applicant: CARDIAI TECHNOLOGIES LTD., Calgary (CA)

(72) Inventors: Anmol KAPOOR, Calgary (CA); Sidharth Singh BHINDER, Calgary (CA)

(21) Appl. No.: 19/476,826

(22) PCT Filed: Apr. 19, 2024
(86) PCT No.: PCT/CA2024/050513

§ 371 Date: Oct. 20, 2025

Related U.S. Application Data
(60) Provisional application No. 63/460,692, filed on Apr. 20, 2023.

(51) Int. Cl. G16H 50/20; G16B 25/10; G16B 40/00; G16H 10/40

(52) U.S. Cl. CPC G16H 50/20; G16B 25/10; G16B 40/00; G16H 10/40

(57) ABSTRACT

A health-analysis method has the steps of: obtaining testing data of a patient's sample, cleaning the testing data by removing data items unrelated to the health analysis, obtaining statistics of the cleaned testing data, and obtaining analysis results of one or more genes and/or species of the patient's sample using one or more artificial intelligence (AI) methods based on the obtained statistics of the cleaned testing data and statistics of healthy data.

### CROSS-REFERENCE TO RELATED APPLICATIONS

This application claims the benefit of U.S. Provisional Patent Application Ser. No. 63/460,692, filed Apr. 20, 2023, the content of which is incorporated herein by reference in its entirety.

### FIELD OF THE DISCLOSURE

The present disclosure relates generally to system and method for analyzing, monitoring, and/or predicting people's health conditions, and in particular to system and method for analyzing and/or monitoring people's health conditions based on pharmacogenomics and/or microbiome.

### BACKGROUND

There is an increasing need for analyzing, monitoring, and/or predicting people's health conditions. In addition to traditional means such as monitoring one's heart rate, blood pressure, and/or the like, other means may also be used.

For example, human genomic data carries unique information about an individual and offers unprecedented opportunities for healthcare. The clinical interpretations derived from large genomic datasets may greatly improve healthcare and pave the way for personalized medicine. Thus, pharmacogenomics may be used for analyzing, monitoring, and/or predicting people's health conditions.

As another example, microbiome is the collection of the microbes such as bacteria, fungi, viruses, and their genes, that naturally live on and in human bodies. As a key interface between the body and the environment, microbiome may affect human health in many ways. Monitoring one's microbiome may also help in analyzing, monitoring, and/or predicting people's health conditions.

### SUMMARY

According to one aspect of this disclosure, there is provided a health-analysis method comprising: obtaining testing data of a patient's sample; cleaning the testing data by removing data items unrelated to the health analysis; obtaining statistics of the cleaned testing data; and obtaining analysis results of one or more genes and/or species of the patient's sample using one or more artificial intelligence (AI) methods based on the obtained statistics of the cleaned testing data and statistics of healthy data.

In some embodiments, the health-analysis method further comprises: collecting the testing data using one or more sensors.

In some embodiments, said obtaining the analysis results of the one or more genes and/or species of the patient's sample comprises: using a machine learning method for identifying characteristics of the one or more genes, and/or for analyzing genetic data of the testing data and identifying patterns or relationships between different genetic markers.

In some embodiments, said obtaining the analysis results of the one or more genes and/or species of the patient's sample comprises: using a random forest method for building a plurality of decision trees and taking their majority vote for classification.

In some embodiments, said obtaining the analysis results of the one or more genes and/or species of the patient's sample comprises: using a regression method for predicting expression of the one or more genes.

In some embodiments, said obtaining the analysis results of the one or more genes and/or species of the patient's sample comprises: using a clustering method for grouping individuals based on their genetic similarity.

In some embodiments, said obtaining the analysis results of the one or more genes and/or species of the patient's sample comprises: using a maximum relevance minimum redundancy (MRMR) method for selecting a set of features of minimum redundancy and maximum relevance with respect to a target variable.

In some embodiments, said obtaining the analysis results of the one or more genes and/or species of the patient's sample comprises: using a deep learning method for analyzing datasets of the testing data and identifying features and/or patterns.

In some embodiments, said obtaining the analysis results of the one or more genes and/or species of the patient's sample comprises: using a convolutional neural network (CNN) for recognizing patterns in gene expression data of the testing data for identifying one of the one or more genes that is associated with an environmental condition.

In some embodiments, said obtaining the analysis results of the one or more genes and/or species of the patient's sample comprises: using a reinforcement learning method for optimizing genetic datasets by identifying informative genetic features.

In some embodiments, said obtaining the analysis results of the one or more genes and/or species of the patient's sample comprises: using a reinforcement learning method for selecting informative genetic markers for a population while minimizing a number of genetic markers required for predictions.

In some embodiments, said obtaining the analysis results of the one or more genes and/or species of the patient's sample comprises: using a genetic method for optimizing genetic datasets by iteratively testing and refining different combinations of genetic markers.

In some embodiments, said obtaining the analysis results of the one or more genes and/or species of the patient's sample comprises: using a genetic method for identifying an optimal set of genetic markers for predicting a risk of a particular disease or condition.

In some embodiments, said cleaning the testing data comprises: cleaning the testing data using one or more gene panels related to the health analysis.

In some embodiments, said obtaining the statistics of the cleaned testing data comprises: obtaining standard deviations and/or variance of the cleaned testing data.

In some embodiments, said obtaining the standard deviations and/or variance of the cleaned testing data with respect to the healthy data comprises: obtaining the standard deviations and/or variance of real read, estimated read, and abundance of the one or more genes from the cleaned testing data.

In some embodiments, said obtaining the one or more genes and/or species analysis results comprises: using the one or more AI models to obtain analysis results regarding similarity between the cleaned testing data and the healthy data and/or ranking of the one or more genes or bacteria based on the obtained statistics.

In some embodiments, the health-analysis method further comprises: automatically monitoring development in one or more research areas; and automatically notifying patients and/or healthcare workers regarding changes of functions of the one or more genes and/or changes of ranking of the one or more genes.

In some embodiments, the health-analysis method further comprises: using the obtained analysis results of the one or more genes and/or species of the patient's sample for determining one or more diseases for the patient.

In some embodiments, the health-analysis method further comprises: using the obtained analysis results of the one or more genes and/or species of the patient's sample for analyzing and and/or monitoring the patient's response to drugs.

According to one aspect of this disclosure, there is provided one or more processors for performing the above-described method.

According to one aspect of this disclosure, there is provided an apparatus comprising one or more processors; and one or more non-transitory computer-readable storage devices functionally coupled to the one or more processors; the one or more non-transitory computer-readable storage devices comprising computer-executable instructions, wherein the instructions, when executed, cause the one or more processors to perform the above-described method.

According to one aspect of this disclosure, there is provided one or more non-transitory computer-readable storage devices comprising computer-executable instructions, wherein the instructions, when executed, cause a processing structure such as one or more processors to perform the above-described method.

### BRIEF DESCRIPTION OF THE DRAWINGS

For a more complete understanding of the disclosure, reference is made to the following description and accompanying drawings, in which:

FIG. 1 is a schematic diagram of a computer network system for data sharing, according to some embodiments of the present disclosure;

FIG. 2 is a schematic diagram showing a simplified hardware structure of a computing device of the computer network system shown in FIG. 1;

FIG. 3 a schematic diagram showing a simplified software architecture of a computing device of the computer network system shown in FIG. 1;

FIG. 4 is a schematic diagram showing the functional structure of the computer network system shown in FIG. 1 for genetic monitoring, according to some embodiments of this disclosure;

FIG. 5 is a flowchart showing a health-analysis process executed by the computer network system shown in FIG. 1, according to some embodiments of this disclosure; and

FIG. 6 is a schematic diagram showing the functional structure of the computer network system shown in FIG. 1 for pharmacogenomics monitoring, according to some embodiments of this disclosure.

### DETAILED DESCRIPTION

### A. System Structure

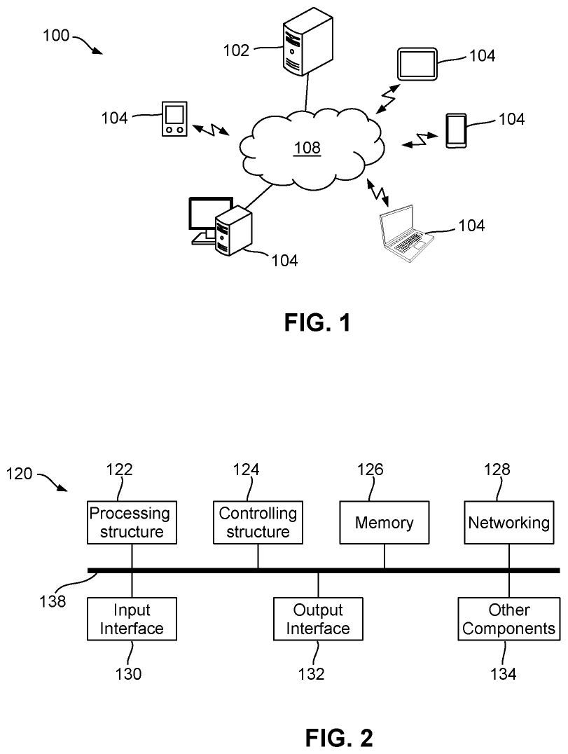

Turning now to FIG. 1, a computer network system is shown and is generally identified using reference numeral 100. In various embodiments, the computer network system 100 may be used for analyzing and/or monitoring people's health conditions based on pharmacogenomics and/or microbiome.

As shown, the computer network system 100 comprises one or more server computers 102 and a plurality of client computing devices 104 functionally interconnected by a network 108, such as the Internet, a local area network (LAN), a wide area network (WAN), a metropolitan area network (MAN), and/or the like, via suitable wired and/or wireless networking connections.

The server computers 102 may be computing devices designed specifically for use as a server, and/or general-purpose computing devices acting as server computers while also being used by various users. Each server computer 102 may execute one or more server programs.

The client computing devices 104 may be portable and/or non-portable computing devices such as laptop computers, tablets, smartphones, Personal Digital Assistants (PDAs), desktop computers, and/or the like. Each client computing device 104 may execute one or more client application programs which sometimes may be called “apps”.

Generally, the computing devices 102 and 104 have a similar hardware structure such as a hardware structure 120 shown in FIG. 2. As shown, the computing device 102/104 comprises a processing structure 122, a controlling structure 124, one or more non-transitory computer-readable memory or storage devices 126, a network interface 128, an input interface 130, and an output interface 132, functionally interconnected by a system bus 138. The computing device 102/104 may also comprise other components 134 coupled to the system bus 138.

The processing structure 122 may be one or more single-core or multiple-core computing processors such as INTEL® microprocessors (INTEL is a registered trademark of Intel Corp., Santa Clara, CA, USA), AMD® microprocessors (AMD is a registered trademark of Advanced Micro Devices Inc., Sunnyvale, CA, USA), ARM® microprocessors (ARM is a registered trademark of Arm Ltd., Cambridge, UK) manufactured by a variety of manufactures such as Qualcomm of San Diego, California, USA, under the ARM® architecture, or the like. When the processing structure 122 comprises a plurality of processors, the processors thereof may collaborate via a specialized circuit such as a specialized bus or via the system bus 138.

The processing structure 122 may also comprise one or more real-time processors, programmable logic controllers (PLCs), microcontroller units (MCUs), u-controllers (UCs), specialized/customized processors and/or controllers using, for example, field-programmable gate array (FPGA) or application-specific integrated circuit (ASIC) technologies, and/or the like.

Generally, each processor of the processing structure 122 comprises necessary circuitries implemented using technologies such as electrical and/or optical hardware components for executing one or more processes to perform various tasks. In many embodiments, the one or more processes may be implemented as firmware and/or software stored in the memory 126. Those skilled in the art will appreciate that, in these embodiments, the one or more processors of the processing structure 122, are usually of no use without meaningful firmware and/or software.

The controlling structure 124 comprises one or more controlling circuits, such as graphic controllers, input/output chipsets, and the like, for coordinating operations of various hardware components and modules of the computing device 102/104.

The memory 126 comprises one or more one or more non-transitory computer-readable storage devices or media accessible by the processing structure 122 and the controlling structure 124 for reading and/or storing instructions for the processing structure 122 to execute, and for reading and/or storing data, including input data and data generated by the processing structure 122 and the controlling structure 124. The memory 126 may be volatile and/or non-volatile, non-removable or removable memory such as RAM, ROM, EEPROM, solid-state memory, hard disks, CD, DVD, flash memory, or the like. In use, the memory 126 is generally divided into a plurality of portions for different use purposes. For example, a portion of the memory 126 (denoted as storage memory herein) may be used for long-term data storing, for example, for storing files or databases. Another portion of the memory 126 may be used as the system memory for storing data during processing (denoted as working memory herein).

The network interface 128 comprises one or more network modules for connecting to other computing devices or networks through the network 108 by using suitable wired and/or wireless communication technologies such as Ethernet, WI-FI® (WI-FI is a registered trademark of Wi-Fi Alliance, Austin, TX, USA), BLUETOOTH® (BLUETOOTH is a registered trademark of Bluetooth Sig Inc., Kirkland, WA, USA), Bluetooth Low Energy (BLE), Z-Wave, Long Range (LoRa), ZIGBEE® (ZIGBEE is a registered trademark of ZigBee Alliance Corp., San Ramon, CA, USA), wireless broadband communication technologies such as Global System for Mobile Communications (GSM), Code Division Multiple Access (CDMA), Universal Mobile Telecommunications System (UMTS), Worldwide Interoperability for Microwave Access (WiMAX), CDMA2000, Long Term Evolution (LTE), 3GPP, 5G New Radio (5G NR) and/or other 5G networks, and/or the like. In some embodiments, parallel ports, serial ports, USB connections, optical connections, or the like may also be used for connecting other computing devices or networks although they are usually considered as input/output interfaces for connecting input/output devices.

The input interface 130 comprises one or more input modules for one or more users to input data via, for example, touch-sensitive screens, touch-sensitive whiteboards, touch-pads, keyboards, computer nice, trackballs, microphones, scanners, cameras, and/or the like. The input interface 130 may be a physically integrated part of the computing device 102/104 (for example, the touch-pad of a laptop computer or the touch-sensitive screen of a tablet), or may be a device physically separated from but functionally coupled to, other components of the computing device 102/104 (for example, a computer mouse). The input interface 130, in some implementation, may be integrated with a display output to form a touch-sensitive screen or a touch-sensitive whiteboard.

The output interface 132 comprises one or more output modules for output data to a user. Examples of the output modules include displays (such as monitors, LCD displays, LED displays, projectors, and the like), speakers, printers, virtual reality (VR) headsets, augmented reality (AR) goggles, and/or the like. The output interface 132 may be a physically integrated part of the computing device 102/104 (for example, the display of a laptop computer or a tablet), or may be a device physically separate from but functionally coupled to other components of the computing device 102/104 (for example, the monitor of a desktop computer).

The computing device 102/104 may also comprise other components 134 such as one or more positioning modules, temperature sensors, barometers, inertial measurement units (IMUs), and/or the like. Examples of the positioning modules may be one or more global navigation satellite system (GNSS) components (for example, one or more components for operation with the Global Positioning System (GPS) of USA, Global'naya Navigatsionnaya Sputnikovaya Sistema (GLONASS) of Russia, the Galileo positioning system of the European Union, and/or the Beidou system of China).

The system bus 138 interconnects various components 122 to 134 enabling them to transmit and receive data and control signals to and from each other.

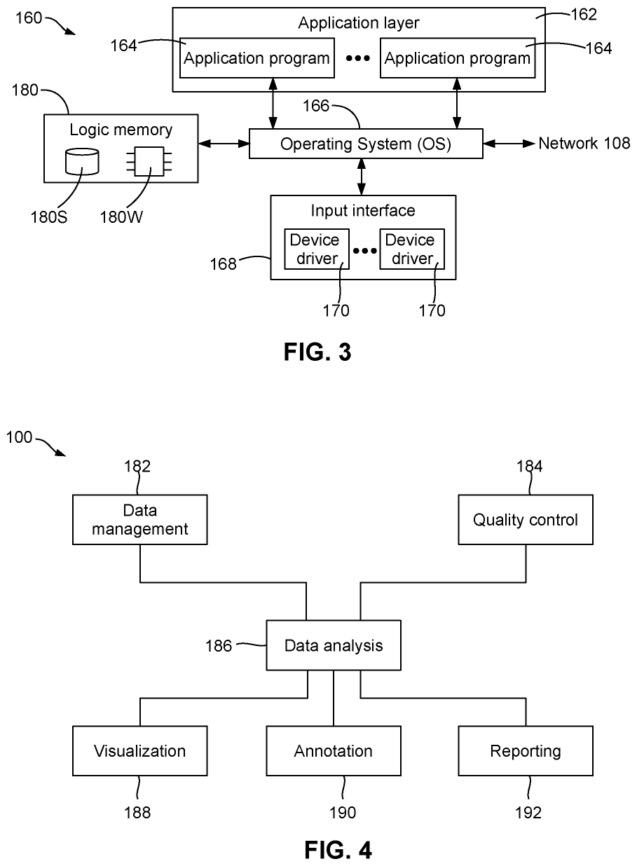

FIG. 3 shows a simplified software architecture 160 of the computing device 102 or 104. The software architecture 160 comprises an application layer 162, an operating system 166, a logical input/output (I/O) interface 168, and a logical memory 172. The application layer 162, operating system 166, and logical I/O interface 168 are generally implemented as computer-executable instructions or code in the form of software programs or firmware programs stored in the logical memory 172 which may be executed by the processing structure 122.

Herein, a software or firmware program is a set of computer-executable instructions or code stored in one or more non-transitory computer-readable storage devices or media such as the memory 126, and may be read and executed by the processing structure 122 and/or other suitable components of the computing device 102/104 for performing one or more processes. Those skilled in the art will appreciate that a program may be implemented as either software or firmware, depending on the design purposes and requirements. Therefore, for ease of description, the terms “software” and “firmware” may be interchangeably used hereinafter.

Referring back to FIG. 3, the application layer 162 comprises one or more application programs 164 executed by or performed by the processing structure 122 for performing various tasks.

The operating system 166 manages various hardware components of the computing device 102 or 104 via the logical I/O interface 168, manages the logical memory 172, and manages and supports the application programs 164. The operating system 166 is also in communication with other computing devices (not shown) via the network 108 to allow the application programs 164 to communicate with programs running on other computing devices. As those skilled in the art will appreciate, the operating system 166 may be any suitable operating system such as MICROSOFT® WINDOWS® (MICROSOFT and WINDOWS are registered trademarks of the Microsoft Corp., Redmond, WA, USA), APPLE® OS X, APPLE® iOS (APPLE is a registered trademark of Apple Inc., Cupertino, CA, USA), Linux, ANDROID® (ANDROID is a registered trademark of Google Inc., Mountain View, CA, USA), or the like. The computing devices 102 and 104 of the computer network system 100 may all have the same operating system, or may have different operating systems.

The logical I/O interface 168 comprises one or more device drivers 170 for communicating with respective input and output interfaces 130 and 132 for receiving data therefrom and sending data thereto. Received data may be sent to the application layer 162 for being processed by one or more application programs 164. Data generated by the application programs 164 may be sent to the logical I/O interface 168 for outputting to various output devices (via the output interface 132). The logical memory 172 is a logical mapping of the physical memory 126 for facilitating the application programs 164 to access. In this embodiment, the logical memory 172 comprises a storage memory area that may be mapped to a non-volatile physical memory such as hard disks, solid-state disks, flash drives, and/or the like, generally for long-term data storage therein. The logical memory 172 also comprises a working memory area that is generally mapped to high-speed, and in some implementations, volatile physical memory such as RAM, generally for application programs 164 to temporarily store data during program execution. For example, an application program 164 may load data from the storage memory area into the working memory area, and may store data generated during its execution into the working memory area. The application program 164 may also store some data into the storage memory area as required or in response to a user's command.

In a server computer 102, the application layer 162 generally comprises one or more server-side application programs 164 which provide(s) server functions for managing network communication with client computing devices 104 and facilitating collaboration between the server computer 102 and the client computing devices 104. Herein, the term “server” may refer to a server computer 102 from a hardware point of view, or to a logical server from a software point of view, depending on the context.

As described above, the processing structure 122 is usually of no use without meaningful firmware and/or software. Similarly, while the computer network system 100 may have the potential to perform various tasks, it cannot perform any tasks and is of no use without meaningful firmware and/or software. As will be described in more detail later, the computer network system 100 described herein, as a combination of hardware and software, generally produces tangible results tied to the physical world, wherein the tangible results such as those described herein may lead to improvements to the computer and system themselves.

As will be described in more detail below, in various embodiments, the computer network system 100 may be used to provide personalized care to individuals with integration of Internet-of-things (IOT), wearable devices such as smartwatches and/or smart-bands, mobile health devices, telehealth, clinical consultations, and/or the like to provide prediction and management of various health conditions such as cardiology, oncology, mental health, gastrointestinal (GI), and/or the like. The computer network system 100 thus allows users to develop personalized pharmaceutical and nutraceutical recommendations, and get related products to people in a timely manner.

### B. Health Analysis and Monitoring

In some embodiments, the computer network system 100 may be used as a genetic-monitoring system for analyzing and and/or monitoring people's health conditions based on genetic analysis (including human genes analysis and/or microbiome genetic analysis).

In some embodiments, the genetic-monitoring system 100 may function as a health-alarm system and use one or more artificial intelligence (AI) methods (such as AI algorithms) and models to monitor individual's genes in real-time by analyzing the individual's whole-exome sequencing (WES), whole genome sequencing (WGS), and/or sequencing data (such data may be obtained from other suitable systems).

FIG. 4 is a schematic diagram showing the functional structure of the genetic-monitoring system 100, according to some embodiments of this disclosure. As shown, the genetic-monitoring system 100 may comprise a data management module 182 and a quality control module 184 for supporting a data analysis module 186. The genetic-monitoring system 100 may comprise a visualization module 188, an annotation module 190, and a reporting module 192 for interfacing the users with the analysis results obtained from the data analysis module 186.

The data management module 182 may comprise suitable tools for managing and organizing large datasets of genetic data, including importing raw sequencing data, organizing samples into different populations or groups, storing metadata such as sample collection dates and locations, and/or the like.

The quality control module 184 ensures that the genetic data is accurate and reliable, and may comprise suitable quality-control tools to check for sequencing errors, contamination, or other issues that might affect the data.

The data analysis module 186 may comprise one or more suitable analysis tools to identify patterns in the genetic data such as changes in gene expression or genetic diversity over time. The data analysis module 186 may use suitable statistical methods to identify significant differences between populations, or use suitable AI algorithms such as machine learning methods or algorithms to identify patterns in large datasets.

The visualization module 188 provides interpretation of the genetic data to users, and may comprise one or more suitable tools for visualizing the data in various formats such as heatmaps, scatterplots, phylogenetic trees, and/or the like.

The annotation module 190 may be used to identify novel genes or gene variants, and may comprise suitable tools for annotating the genetic data such as assigning gene names, characterizing their functions, and/or the like.

The reporting module 192 may comprise one or more suitable tools for generating reports or summaries of the genetic data, which may be used to inform publications, management plans, conservation efforts, and/or the like.

With the modules 182 to 192, the genetic-monitoring system 100 may be used for genetic monitoring and analysis in a powerful, flexible, and user-friendly manner to enable everyone to effectively analyze, monitor and interpret complex genetic data.

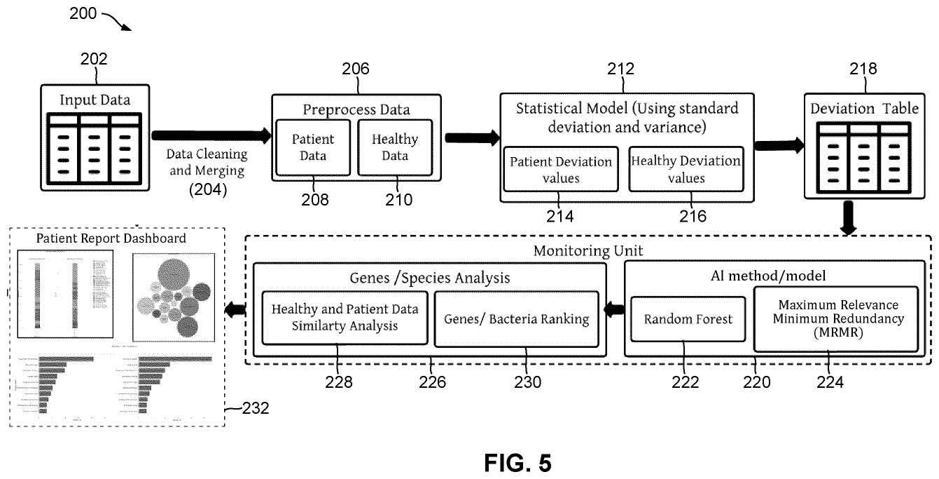

FIG. 5 is a flowchart showing a health-analysis process 200 of the data analysis module 186, executed by the computer network system 100 (or more specifically, one or more processors 122 thereof) for analyzing and/or monitoring people's health conditions.

Before the health-analysis process 200 starts, one or more suitable sensors may be used for collecting testing data of a patient's sample. The collected testing data is then sent to the computer network system 100.

As shown in FIG. 5, the computer network system 100 receives testing data such as testing data of human genes or microbiome data (such as microbiome genetic testing data) as input data 202. As those skilled in the art appreciate, the input data 202 may comprise various data items, and depending on the health conditions to be analyzed, some of the data items may be relevant to the required analysis and others of the data items may be irrelevant to the required analysis. Therefore, the computer network system 100 may clean the input data 202 based on the analysis requirements (step 204). For example, in some embodiments, the computer network system 100 may use a specific gene panel (which comprises one or more genes relevant or otherwise related to a disease) to clean the input data 202 (for example, to remove the irrelevant data items) and obtain cleaned patient data 208. The computer network system 100 then merges the cleaned patient data 208 with relevant healthy data 210 to a single dataset for ease of processing.

At step 206, the computer network system 100 preprocesses the dataset as needed. At step 212, the computer network system 100 processes the dataset by comparing the cleaned patient data 208 with relevant healthy data 210 using a statistical model to calculate, for example, the standard deviation and/or variance of real read, estimated read, and abundance of the genes. In these embodiments, the patient deviation values 214 and the healthy deviation values 216 are obtained, and are merged into a deviation table 218.

The computer network system 100 then uses a suitable AI method 220 and a corresponding AI model to extract features from the deviation table 218. The AI method/model 220 comprises a suitable machine learning method/model 222 for classification and a suitable feature selection method 224 for identifying characteristics of genes.

In some embodiments, the machine learning method 222 may comprise a random forest method which builds a plurality of decision trees on different samples and takes their majority vote for classification and average for regression to predict the expression of specific genes.

In some embodiments, the feature selection method 224 may be the maximum relevance minimum redundancy (MRMR) method, which selects an optimal set of mutually and maximally dissimilar features (that is, features of minimum redundancy and maximum relevance with respect to the target variable).

The output of the AI method 220 is then used for analysis of genes and/or species (step 226), such as the healthy and patient data similarity analysis 228 and/or the genes/bacteria ranking 230. The analysis results are then reported to one or more users such as the patient in suitable format (step 232) such as using a patient report dashboard to present the analysis results in one or more graphics.

By using one or more AI models and related AI methods/algorithms, the genetic-monitoring system 100 may create genetic datasets by analyzing and processing large amounts of genetic data in an automated and efficient manner.

In some embodiments, the genetic-monitoring system 100 may use machine learning algorithms to analyze genetic data and identify patterns or relationships between different genetic markers or traits. For example, the genetic-monitoring system 100 may use clustering algorithms to group individuals based on their genetic similarity, while using regression algorithms to predict the expression of specific genes based on other genetic features.

In some embodiments, the genetic-monitoring system 100 may use deep learning algorithms such as convolutional neural networks (CNNs) to analyze large datasets of genetic data and identify important features and/or patterns. For example, CNNs may be trained to recognize specific patterns in gene expression data, which may help to identify genes that are associated with specific phenotypic traits or environmental conditions.

In some embodiments, the genetic-monitoring system 100 may use reinforcement learning algorithms to optimize genetic datasets by identifying the most informative or relevant genetic features. For example, reinforcement learning algorithms may be used to select the most informative genetic markers for a particular population or trait, while minimizing the number of markers needed to achieve accurate predictions.

In some embodiments, the genetic-monitoring system 100 may use genetic algorithms to optimize genetic datasets by iteratively testing and refining different combinations of genetic markers. For example, genetic algorithms may be used to identify the optimal set of genetic markers for predicting the risk of a particular disease or condition.

By using the one or more AI algorithms and AI models, the genetic-monitoring system 100 may enlarge the datasets as soon as new dataset is detected. The genetic-monitoring system 100 may check individual's data and put an alarm to the individual as needed.

In some embodiments, the genetic-monitoring system 100 may also automatically monitor and learn the development in relevant research areas. For example, when genes functions changes or ranking of gene changes, the genetic-monitoring system 100 may automatically update or notify patients and/or healthcare workers about the risks.

In these embodiments, the genetic-monitoring system 100 automatically and repeatedly monitors various genetic research resources such as various genetic-related publications and literatures, and retrieves updated or newly developed genetic research results. The data management module 182 also checks the types of genomic data of various patients. If the retrieved genetic research results are relevant to the types of genomic data of a patient, the data management module 182 may send an alarm or notification to the patient and/or relevant healthcare workers to notify them the updated or newly developed genetic research results.

For example, once the WGS, WES, and/or, microbiome data of a patient is obtained, the genetic-monitoring system 100 may analyze the obtained data and link it with relevant genetic research results obtained from the automatically and repeatedly monitored genetic research resources, and notify the patient and/or relevant healthcare workers regarding the genetic research results and/or the potential impact to the patient.

In some embodiments, the genetic-monitoring system 100 may provide subscription-based services and may be linked with telemedicine services for providing improved treatment plans, drugs and diagnostic evaluations, and/or the like in the lifespan of a patient.

As described above, the computer network system 100 may perform various functions based on genetic analysis. As those skilled in the art will appreciate, genetic analysis may involve the identification of a set of genes (denoted a gene panel) related to a disease (such as having the potential to trigger the disease and/or having the potential for identifying targeted drugs to target the disease). Below lists some diseases that may be determined by the computer network system 100 and the genetic analysis thereof through identification of related gene panels.
Abnormal pulmonary interstitial morphology, which is a condition where there is abnormal tissue growth in the lungs, leading to breathing difficulties and a decreased ability to oxygenate the blood.
Absence of the septum pellucidum, which is a rare congenital condition in which the thin membrane that separates the two sides of the brain does not form properly.
Attention deficit hyperactivity disorder (ADHD), which is a neurodevelopmental disorder that affects behavior, impulse control, and attention span.
Adrenocortical carcinoma, which is a rare cancer that develops in the adrenal gland, which produces hormones that regulate the body's metabolism and response to stress.
Adult-onset movement disorder, which is a mutation-related genetic disease, such as Huntington's disease and spinocerebellar ataxia.
Adult solid tumours cancer, which is caused by an inherited genetic condition that increases the risk of developing certain types of cancer in adulthood.
Adult solid tumors for rare disease, which includes a group of rare cancers that can affect various organs and tissues in the body.
Agnosia, which is a neurological condition that impairs a person's ability to recognize or identify objects, people, or sounds, despite having normal sensory abilities.
Aicardi goutieres syndrome disorder, which is a rare genetic disorder that affects the brain and the immune system, causing developmental delay, seizures, and other neurological symptoms.
Alexander disease, which is a rare genetic disorder that affects the myelin, or the protective covering, of nerve fibers in the brain. This can lead to developmental delay, seizures, and a progressive loss of developmental milestones.
Amyotrophic Lateral Sclerosis (ALS), also known as Lou Gehrig's disease, which is a progressive neurodegenerative disease that affects nerve cells in the brain and spinal cord responsible for controlling voluntary muscles, resulting in muscle weakness and atrophy, and eventually leading to paralysis. The cause is largely unknown, and there is currently no cure.
Amyloidosis, which is a group of rare diseases in which abnormal proteins called amyloid accumulate in different tissues and organs throughout the body, affecting their normal structure and function. These proteins can accumulate in various organs such as the heart, kidneys, liver, and nervous system. There are several types of amyloidosis, including primary, secondary, and hereditary forms. Symptoms of amyloidosis may vary depending on the affected organs, but can include fatigue, weight loss, swelling, shortness of breath, and abnormal heart rhythm. Diagnosis of amyloidosis is usually made through a combination of clinical exams, imaging tests, and tissue biopsy to confirm the presence of amyloid protein. Treatment for amyloidosis depends on the type and severity of the condition, but may include medication, chemotherapy, stem cell transplantation, or organ transplantation in severe cases. Prognosis for amyloidosis can vary depending on the type and stage of the disease, as well as the effectiveness of treatment. Early diagnosis and management are important for improving outcomes and quality of life for affected individuals.
Anemia, which is a condition in which there is a shortage of red blood cells, leading to fatigue, weakness, and shortness of breath.
Anencephaly, which is a rare birth defect where the brain and skull do not develop properly during pregnancy. Babies with anencephaly are usually stillborn or die shortly after birth.
Antinuclear antibodies (ANA), which is a type of autoantibody that target the nucleus of cells. ANA is produced by the immune system in response to perceived threats, such as infections or cancer, but can also be produced in autoimmune diseases. ANA testing is commonly used to diagnose autoimmune diseases such as lupus and Sjögren's syndrome. A positive ANA test alone is not diagnostic of an autoimmune disease, as it can also be found in healthy individuals or those with infections. Further testing and evaluation is required to confirm a diagnosis.
Anxiety and panic attacks, which is a mental health disorder characterized by excessive fear or worry, often leading to panic attacks.
Aphasia, which is a language disorder that affects a person's ability to communicate effectively. It is typically caused by damage to the language centers of the brain, often as a result of stroke, traumatic brain injury, or neurological disorders such as Alzheimer's disease. Aphasia can affect a person's ability to speak, understand language, read, write, or use gestures. The severity of the symptoms can vary widely, from mild difficulty finding words to complete loss of language abilities. Treatment for aphasia may include speech therapy, which focuses on improving communication skills through exercises that target specific language functions, as well as compensatory strategies such as the use of communication aids or alternative forms of communication. The goal of treatment is to improve the person's ability to communicate and participate in daily activities, as well as to support their overall quality of life.
Apraxia, which is a neurological condition that affects a person's ability to plan and carry out voluntary movements or activities, even though they have the physical ability to do so. This can affect various aspects of daily life, such as speech, dressing, and eating. Apraxia can be caused by damage to the brain, such as from a stroke, traumatic brain injury, or degenerative neurological conditions. Symptoms of apraxia may include difficulty initiating movements, performing gestures or tasks in the correct sequence, and imitating movements or sounds. Diagnosis of apraxia is usually made through a combination of clinical exams, imaging tests, and assessments of motor skills and language abilities. Treatment for apraxia may involve speech therapy, occupational therapy, and other forms of rehabilitation that focus on improving motor planning and coordination. The prognosis for apraxia varies depending on the underlying cause, the severity of the condition, and the effectiveness of treatment.
Arrhythmogenic cardiomyopathy (AC), which is a rare genetic disorder that affects the heart muscle and can cause abnormal heart rhythms and heart failure. It is characterized by the replacement of normal heart muscle tissue with fatty and fibrous tissue, which can disrupt the normal electrical signals that regulate the heart's rhythm. AC is often diagnosed in young adults, but can affect individuals of any age. Symptoms of AC may include palpitations, chest pain, shortness of breath, and fainting spells. Diagnosis of AC is typically made through a combination of clinical exams, imaging tests, and genetic testing. Treatment for AC focuses on managing symptoms and preventing complications, and may include medications to control heart rhythms, implantable devices such as pacemakers or defibrillators, and lifestyle modifications such as avoiding strenuous exercise. The prognosis for individuals with AC varies depending on the severity of the condition, the presence of associated complications, and the effectiveness of treatment. Regular monitoring and management of AC are important for improving outcomes and preventing complications such as sudden cardiac arrest.
Arteriosclerosis, which is a condition in which the arteries become thick and stiff, leading to a reduced flow of blood to the organs and tissues of the body. This can increase the risk of heart attack, stroke, and other cardiovascular problems. Arteriosclerosis is often caused by a buildup of plaque, which is a combination of cholesterol, fat, and other substances, on the walls of the arteries. Other factors that can contribute to arteriosclerosis include high blood pressure, diabetes, smoking, and a sedentary lifestyle. Treatment for arteriosclerosis may include lifestyle changes such as diet and exercise, medications to manage underlying conditions, and in some cases, surgical interventions such as angioplasty or bypass surgery to improve blood flow.
Arteriovenous malformation (AVM), which is a rare condition in which there is a tangle of abnormal blood vessels connecting arteries and veins in the brain or spinal cord. These vessels are fragile and can rupture, causing bleeding into the brain or spinal cord. Symptoms of AVM may include headaches, seizures, neurological deficits such as weakness or numbness, and vision or speech problems. Diagnosis of AVM is typically made through a combination of clinical exams, imaging tests, and angiography. Treatment for AVM may involve surgery to remove the malformation, embolization to block the abnormal blood vessels, or radiation therapy to reduce the size of the AVM. The outlook for individuals with AVM varies depending on the severity of the condition, the extent of bleeding or neurological damage, and the effectiveness of treatment. Regular monitoring and management of AVM are important for improving outcomes and preventing complications such as stroke or brain injury.
Arthritis, which is a condition characterized by inflammation and stiffness in the joints, causing pain and reduced mobility. There are many different types of arthritis, including rheumatoid arthritis, osteoarthritis, and psoriatic arthritis.
Asthma, which is a chronic respiratory condition characterized by inflammation and narrowing of the airways, which can cause difficulty breathing, coughing, wheezing, and chest tightness. The condition is typically triggered by exposure to certain allergens, irritants, or physical activity. Treatment for asthma usually involves the use of inhaled medications, such as bronchodilators and corticosteroids, to reduce inflammation and improve breathing. Asthma management also often includes identifying and avoiding triggers, as well as developing an action plan for managing flare-ups or exacerbations.
Astrocytoma, which is a type of brain tumor that originates in the star-shaped cells (astrocytes) that support and nourish nerve cells in the brain. Symptoms may include headache, seizures, and changes in mental status.
Ataxia, which refers to a group of disorders that affect coordination and balance. It can result from damage to the cerebellum or other parts of the nervous system. Symptoms may include unsteady gait, difficulty with fine motor tasks, and slurred speech.
Ataxia telangiectasia (A-T), which is a rare genetic disorder that affects the nervous system, immune system, and other body systems. The condition is caused by mutations in the ATM gene, which provides instructions for making a protein that helps repair damaged DNA. Symptoms of A-T can vary widely, but may include difficulty with coordination and balance, telangiectasias (small red spider-like veins) on the skin and eyes, weakened immune system, increased risk of cancer, and progressive lung disease. There is no cure for A-T, and treatment is largely symptomatic and supportive, with a focus on managing symptoms and preventing complications. People with A-T may benefit from physical and occupational therapy, antibiotics to prevent infections, and genetic counseling to manage the risk of passing on the condition to their children.
Atherosclerosis, which is a condition characterized by the buildup of fatty deposits, cholesterol, and other substances in the walls of arteries. This buildup, called plaque, can narrow and harden the arteries, reducing blood flow to vital organs and tissues throughout the body. Atherosclerosis can affect any artery in the body, but it most commonly affects the coronary arteries that supply blood to the heart, leading to coronary artery disease. Other potential complications of atherosclerosis include stroke, peripheral artery disease, and aneurysms. The exact cause of atherosclerosis is not known, but it is believed to be related to a combination of genetic and environmental factors, such as high blood pressure, high cholesterol, smoking, and obesity. Treatment for atherosclerosis typically involves lifestyle changes, such as eating a healthy diet, exercising regularly, and quitting smoking, as well as medications to manage underlying risk factors. In some cases, surgery or other interventions may be necessary to restore blood flow to affected areas.
Atrial fibrillation, which is an irregular heart rhythm that can increase the risk of stroke, heart failure, and other complications, a serious and often life-threatening condition that occurs when blood flow to the brain is blocked or interrupted.

Autism spectrum disorder (ASD), which is a developmental disability caused by differences in the brain. In some embodiments, ASD may be determined by identifying genetic variants associated with ASD. In some embodiments, the genetic-monitoring system 100 may assess an individual's risk of developing ASD based on specific genetic mutations that are associated with an increased risk.
Avascular necrosis, also known as osteonecrosis, which is a condition in which there is a loss of blood supply to bone tissue, leading to bone death and possible collapse.
Back pain, which is a common condition characterized by pain or discomfort in the back, ranging from mild to severe.
Barth syndrome, which is a rare genetic disorder that affects the heart, muscles, and immune system, causing muscle weakness, fatigue.
Beckwith-Wiedemann syndrome (BWS) and other congenital overgrowth disorders, which are a group of genetic conditions that cause abnormal growth of the body tissues, leading to physical abnormalities and increased risk of certain health problems. BWS is caused by mutations in certain genes that regulate cell growth and division, and it is characterized by a range of symptoms such as overgrowth of body parts, abdominal wall defects, and an increased risk of childhood cancers. Other congenital overgrowth disorders include Sotos syndrome, Weaver syndrome, and Simpson-Golabi-Behmel syndrome, each with their own distinct features and genetic causes. Treatment for BWS and other congenital overgrowth disorders is aimed at managing the symptoms and addressing any associated health problems. This may include surgery to correct physical abnormalities, regular monitoring for cancer and other complications, and genetic counseling for affected individuals and their families.
Behcet disease, which is a rare autoimmune disorder that causes inflammation of blood vessels throughout the body, leading to symptoms such as oral and genital ulcers, skin lesions, and eye inflammation.
Bladder cancer, which is a type of cancer that affects the bladder, which is a muscular sac located in the pelvis that stores urine.
Blepharospasm, which is a condition in which the eyelids involuntarily twitch or spasm.
Bradycardia, which is a condition in which the heart beats slower than normal, leading to symptoms such as fatigue, dizziness, and fainting.
Brain cancer susceptibility, which refers to an increased risk of developing tumors or cancers that originate in the brain or spinal cord. Brain tumors can be benign (non-cancerous) or malignant (cancerous), and they can affect people of all ages. There are many factors that may contribute to the development of brain cancer, including genetic predisposition, exposure to certain toxins or radiation, and medical conditions that affect the immune system or the brain. Symptoms of brain cancer may vary depending on the location and type of tumor, but may include headaches, seizures, weakness or numbness in the limbs, vision or hearing changes, and cognitive or personality changes. Treatment for brain cancer may include surgery, radiation therapy, chemotherapy, or a combination of these treatments. The outlook for brain cancer varies depending on the type and stage of the cancer, as well as the individual's age and overall health. Early detection and prompt treatment are important for improving outcomes and quality of life for affected individuals.
Breast cancer susceptibility, which refers to an increased risk of developing breast cancer, a type of cancer that forms in the tissues of the breast. Risk factors for breast cancer include genetic mutations, family history of breast cancer, age, hormonal factors, lifestyle factors, and exposure to certain chemicals or environmental factors. Symptoms of breast cancer may include breast lumps, breast pain, nipple discharge, changes in breast shape or size, or skin changes on the breast. Diagnosis of breast cancer is typically made through a combination of clinical exams, imaging tests, and biopsy of suspicious tissue. Treatment for breast cancer depends on the type, stage, and location of the cancer, as well as the individual's overall health. Options may include surgery, radiation therapy, chemotherapy, hormone therapy, or a combination of these treatments. Prognosis for breast cancer varies depending on the stage and severity of the cancer, as well as the effectiveness of treatment. Early detection and prompt treatment are important for improving outcomes and quality of life for affected individuals.
Bronchiectasis, which is a chronic lung condition characterized by damaged and widened bronchial tubes (airways) that can lead to breathing difficulties, coughing up mucus, and recurrent lung infections. The damage to the airways often results from an underlying condition such as cystic fibrosis, tuberculosis, or pneumonia, or it can be caused by an inherited genetic condition. Common symptoms of bronchiectasis include coughing up phlegm or mucus, shortness of breath, chest pain, and frequent respiratory infections. Treatment may involve antibiotics to treat infections, airway clearance techniques to help clear mucus from the lungs, bronchodilators to open up the airways, and in some cases, surgery to remove damaged lung tissue or to repair an underlying structural defect. With proper management, many individuals with bronchiectasis can lead a relatively normal life, although the condition can be progressive and may worsen over time.
Bronchitis, which is an inflammation of the bronchial tubes, which carry air to and from the lungs, causing coughing, chest pain, and difficulty breathing.
Burkitt lymphoma, which is a rare and aggressive form of non-Hodgkin lymphoma, a type of cancer that affects the lymphatic system. It is characterized by rapidly growing tumors that usually develop in the abdomen, jaw, or other facial bones. Burkitt lymphoma is most commonly found in children and young adults in Africa, but can also occur in other parts of the world. Symptoms may include fever, night sweats, weight loss, and abdominal pain. Treatment typically involves chemotherapy, and may also include radiation therapy and/or surgery depending on the stage and location of the cancer. The prognosis for Burkitt lymphoma can be good with prompt and aggressive treatment.
Bursitis, which is a condition that involves inflammation of a bursa, which is a small fluid-filled sac that cushions and lubricates the joints. Bursae are found throughout the body, particularly in areas where tendons or muscles rub against bones or other structures. Bursitis can occur in any joint but is most common in the shoulder, elbow, hip, knee, and heel. It is usually caused by repetitive movements, prolonged pressure on the joint, injury, or infection. Symptoms of bursitis may include pain, swelling, stiffness, and tenderness around the affected joint. Treatment options include rest, ice, compression, and elevation (RICE), pain relievers, and physical therapy. In severe cases, aspiration of excess fluid or corticosteroid injections may be recommended, and in rare cases, surgical intervention may be necessary.
Canavan disease, which is a rare genetic disorder that affects the brain and nervous system, causing developmental delays and other symptoms.
Carcinoid tumors, which is a type of neuroendocrine tumor that can occur in various parts of the body, most commonly in the gastrointestinal tract or lungs. These tumors arise from specialized cells called neuroendocrine cells, which produce hormones that regulate various bodily functions. Carcinoid tumors can produce excess amounts of hormones, leading to a variety of symptoms such as flushing, diarrhea, wheezing, and heart palpitations. However, many carcinoid tumors do not produce hormones and may not cause any noticeable symptoms. The exact cause of carcinoid tumors is not well understood, but they are believed to develop due to genetic mutations or other changes in the DNA of the affected cells. Treatment options for carcinoid tumors depend on the location, size, and extent of the tumor, and may include surgery, chemotherapy, radiation therapy, and targeted therapy. Prognosis and survival rates vary depending on the stage and grade of the tumor, as well as the patient's overall health and response to treatment.
Carcinoma, which is a type of cancer that arises from cells in the epithelial tissue, which lines the organs and structures in the body.
Cardiac arrhythmias, which refer to abnormal heart rhythms that can lead to serious complications such as stroke or sudden cardiac arrest. In some embodiments, the genetic-monitoring system 100 may analyze a patient's DNA to identify mutations associated with an increased risk for cardiac arrhythmias.
Cardiomyopathy, which is a condition in which the heart muscle becomes weakened, leading to symptoms such as shortness of breath and fatigue. In some embodiments, the genetic-monitoring system 100 may use a genetic panel to test for mutations associated with various types of cardiomyopathy, including those that present in childhood.
Carpal tunnel syndrome, which is a medical condition that occurs when the median nerve is compressed as it passes through the wrist, causing pain, tingling, and numbness in the hand and fingers.
Cerebellar hypoplasia, which is a neurological disorder that occurs when the cerebellum, the part of the brain responsible for controlling movement and balance, is underdeveloped or smaller than normal. This can result in a range of symptoms such as difficulty with coordination and balance, tremors, nystagmus (involuntary eye movements), and developmental delays. Cerebellar Hypoplasia can be caused by a variety of factors, including genetic mutations, infections during pregnancy, exposure to toxins, or lack of oxygen during birth. The severity of symptoms can vary widely, from mild to severe, and there is currently no cure for Cerebellar Hypoplasia. Treatment options may include physical therapy, occupational therapy, and speech therapy, depending on the individual's specific needs and symptoms.
Cerebral atrophy, which is a condition characterized by the loss of brain cells and shrinkage of brain tissue. This can lead to a range of neurological symptoms, depending on the severity and location of the atrophy. Cerebral atrophy can be caused by a variety of factors, including aging, neurological disorders such as Alzheimer's disease and Parkinson's disease, traumatic brain injury, and infections such as HIV/AIDS. Symptoms of cerebral atrophy may include cognitive impairment, memory loss, mood changes, and difficulty with motor skills such as balance and coordination. Treatment for cerebral atrophy is mainly aimed at managing symptoms and slowing the progression of the underlying condition. This may include medications, physical therapy, occupational therapy, and lifestyle modifications such as regular exercise and a healthy diet. In some cases, surgical interventions such as shunting may also be considered. Early detection and management of cerebral atrophy are important for improving outcomes and providing supportive care to affected individuals and their families.
Cerebral palsy (CP), which is a group of neurological disorders that affect movement, muscle tone, and coordination. It is caused by damage to the developing brain, often occurring before birth or during infancy. The symptoms of CP can vary widely, but may include difficulty with muscle control, reflexes, posture, balance, and coordination. It can also cause problems with speech, vision, hearing, and intellectual disability. There is currently no cure for CP, but treatment options such as physical therapy, occupational therapy, speech therapy, and medication can help manage symptoms and improve quality of life. The outlook for individuals with CP can vary depending on the severity of the condition and the availability of appropriate medical care.
Cerebral vascular malformations, which is an abnormality in the blood vessels in the brain, which can lead to seizures, strokes, and other neurological symptoms.
Cerebral vascular malformations which refer to abnormalities in the blood vessels of the brain that can cause a range of symptoms, including seizures, headaches, and neurological deficits.
Chiari malformation, which is a condition in which brain tissue extends into the spinal canal, causing symptoms such as headaches, neck pain, and problems with balance and coordination.
Childhood onset dystonia or chorea or related movement disorder, which is a movement disorder that typically presents in childhood. In some embodiments, the genetic-monitoring system 100 may determine an individual's risk of developing these disorders based on specific genetic mutations that are associated with an increased risk.

Childhood solid tumors cancer susceptibility, which may be determined using a genetic testing panel that analyzes genes associated with an increased risk of developing solid tumors in children.
Cholangiocarcinoma—(bile duct), which refers to a type of cancer that originates in the bile ducts. Genetic testing can help assess an individual's risk of developing cholangiocarcinoma based on specific genetic mutations that are associated with an increased risk. Chordoma, which is a rare type of cancer that occurs in the bones of the skull and spine.
Chronic cough, which refers to a condition where an individual experiences persistent coughing for an extended period, typically more than 8 weeks. There are various causes of chronic cough, including genetic factors.
Chronic granulomatous disease, which is a rare genetic disorder that affects the immune system and makes individuals more susceptible to bacterial and fungal infections.
Chronic inflammatory demyelinating polyneuropathy (CIDP), which is a neurological disorder that causes muscle weakness, numbness, and tingling due to damage to the peripheral nerves.
Chronic obstructive pulmonary disease (COPD), which is a progressive lung disease that makes it difficult to breathe due to obstruction of airflow.
Chronic pain, which refers to pain that persists for a long time, typically more than 12 weeks, and can be caused by a variety of factors such as injury, inflammation, or nerve damage.
Classical tuberous sclerosis, which is a genetic disorder that causes the growth of noncancerous tumors in various organs of the body, including the brain, heart, kidneys, and lungs. Symptoms may include seizures, developmental delay, and skin changes.
Colorectal cancer pertinent cancer susceptibility, which may be determined by using a genetic panel that tests for mutations associated with an increased risk of colorectal cancer.
Colpocephaly, which is a rare congenital brain abnormality characterized by an abnormal enlargement of the occipital horns of the lateral ventricles, which are cavities in the brain that contain cerebrospinal fluid. This condition can occur in both hemispheres of the brain and is typically diagnosed by brain imaging studies, such as MRI or CT scans. In some cases, colpocephaly may be associated with other neurological or developmental disorders, such as cerebral palsy or intellectual disability. Treatment may involve supportive care to manage associated symptoms and address any underlying conditions, such as physical therapy to manage motor difficulties.
Coma, which is a state of prolonged unconsciousness in which a person is unresponsive to external stimuli and unable to communicate.
Common craniosynostosis syndromes, which are a group of conditions where the sutures (joints) between the bones of the skull close too early, leading to abnormal skull shape and potential problems with brain growth and development.
Confirmed fanconi anemia or bloom syndrome, which is a genetic disorder that affects the production of red and white blood cells, leading to an increased risk of developing cancer and other medical conditions.
Congenital muscular dystrophy, which is a group of genetic disorders that affect the muscles and cause muscle weakness and wasting from birth.
Congenital myasthenia, which is a group of inherited disorders that affect the transmission of nerve impulses to muscles, causing weakness and other symptoms.
Congenital myopathy, which refers to a group of inherited muscle disorders that present in infancy or childhood. Genetic testing can help diagnose congenital myopathy and determine the specific genetic mutation responsible.
COVID-19 viral susceptibility, which may be determined using a genetic panel that tests for mutations associated with susceptibility to severe COVID-19 infection.
Cranioectodermal dysplasia, which is rare genetic disorder that affects the development of bones, skin, hair, and other tissues.
Craniopharyngioma, which is a rare brain tumor that develops near the pituitary gland, causing a range of symptoms including headaches, vision loss, and hormonal imbalances. Craniosynostosis, which is a condition in which the bones of a baby's skull fuse prematurely, causing an abnormally shaped head and potential developmental delays.
Cutaneous T-cell lymphoma, which is a type of non-Hodgkin lymphoma that affects the skin and causes itchy, red rashes or patches.
Cystic fibrosis, which is a genetic disorder that affects the lungs and digestive system, causing breathing difficulties, frequent infections, and poor nutrient absorption.
Cytopenia, which is a condition in which there is a lower than normal number of blood cells, but it is not caused by Fanconi anemia, which is a genetic disorder that affects bone marrow.
Dementia, which is a syndrome characterized by a progressive decline in cognitive function and memory, often accompanied by behavioral and psychological symptoms. Dementia can affect people of all ages, but it is more commonly associated with aging. The most common cause of dementia is Alzheimer's disease, but there are other types of dementia, such as vascular dementia, Lewy body dementia, and frontotemporal dementia. Symptoms of dementia may include memory loss, difficulty with language and communication, confusion, mood swings, and changes in personality or behavior. Diagnosis of dementia is usually made through a combination of clinical exams, imaging tests, and cognitive assessments. Treatment for dementia aims to manage symptoms and slow the progression of the disease. This may include medications, occupational therapy, and lifestyle modifications such as regular exercise and a healthy diet. In some cases, supportive care such as counseling and social support may also be helpful. Early detection and management of dementia are important for improving outcomes and quality of life for affected individuals and their families.
Dementia with Lewy bodies (DLB), which is a progressive brain disorder that primarily affects cognitive and motor functions. It is characterized by the abnormal buildup of a protein called alpha-synuclein in the brain, which leads to the formation of Lewy bodies, abnormal protein clumps that cause damage to brain cells. Symptoms of DLB can include fluctuating cognitive impairment, visual hallucinations, movement disorders such as tremors and stiffness, and sleep disturbances. Treatment typically involves medications to manage symptoms, such as cholinesterase inhibitors for cognitive impairment and dopamine agonists for movement disorders.
Depression, which is a mood disorder characterized by persistent feelings of sadness, hopelessness, and a loss of interest in activities.
Developmental and epileptic encephalopathy (DEE), also known as Ohtahara syndrome, which is a rare genetic disorder that affects brain development and causes seizures in infants. Symptoms typically appear in the first few weeks of life and can include seizures, developmental delays, low muscle tone, and breathing difficulties. DEE is caused by mutations in genes that play a role in brain development and function. Diagnosis is made through a combination of clinical exams, imaging tests, and genetic testing. Treatment for DEE is focused on managing symptoms and may include medications to control seizures, physical therapy, and supportive care for breathing and feeding difficulties. The prognosis for DEE varies depending on the individual and the severity of the condition, but it is generally considered to be a severe and life-limiting condition. Early detection and management of DEE are important for improving outcomes and providing supportive care to affected individuals and their families.
Dilated cardiomyopathy and conduction defects, which is a genetic condition that can cause heart failure and other cardiac problems.
Dysautonomia (abnormal autonomic nervous system physiology), which is a condition in which the autonomic nervous system, which controls involuntary functions such as heart rate, blood pressure, and digestion, does not function properly.
Dysgraphia, which is a learning disorder that affects a person's ability to write legibly and effectively.
Dyslexia, which refers to a condition where an individual has difficulty reading, writing, and spelling despite adequate intelligence and educational opportunities. There are various causes of dyslexia, including genetic factors.
Early onset dementia (encompassing fronto-temporal dementia and prion disease), which refers to a group of neurological disorders that cause progressive cognitive decline and affect individuals before the age of 65. This includes frontotemporal dementia (FTD) and prion disease. FTD is a type of dementia that affects the front and sides of the brain, leading to changes in behavior, personality, and language abilities. Prion diseases, on the other hand, are caused by the accumulation of abnormal proteins in the brain, leading to neurological symptoms such as dementia, movement disorders, and seizures. The symptoms of early onset dementia can vary widely, depending on the underlying cause, but may include memory loss, difficulty with language, impaired judgment, and changes in personality or behavior. There is currently no cure for early onset dementia, and treatment options are mainly aimed at managing symptoms and improving quality of life. This may include medications to manage behavioral and mood changes, as well as speech and language therapy to help with communication difficulties. Early diagnosis and management of early onset dementia are important for improving outcomes and providing support to affected individuals and their families.
Encephalopathy, which is a general term that refers to a brain dysfunction or disease that affects cognitive function, behavior, and personality.
Endocarditis, which is an infection of the inner lining of the heart, often caused by bacteria entering the bloodstream.
Endocrine neoplasms, which is a group of rare tumors that affect the endocrine system, which is responsible for producing hormones in the body.
Endometrial cancer pertinent cancer susceptibility, which may be determined by using a genetic test that assesses an individual's risk for developing endometrial cancer based on inherited gene mutations that are associated with this type of cancer.
Ependymoma, which is a type of tumor that can develop in the brain or spinal cord and can cause headaches, nausea, and changes in vision or hearing.
Ewing sarcoma, which is a rare type of bone or soft tissue cancer that can cause pain, swelling, and other symptoms.
Facial Palsy, which is a condition characterized by weakness or paralysis of the muscles of the face, typically on one side. It may be caused by a viral infection, trauma, or other factors.
Familial breast cancer, which is a genetic disorder that increases the risk of developing breast cancer in individuals with a family history of the disease.
Familial dysautonomia (FD), also known as Riley-Day syndrome, is a rare genetic disorder that affects the autonomic nervous system. It is most common in people of Ashkenazi Jewish descent. The condition is caused by mutations in the IKBKAP gene, which is responsible for producing a protein that is important for the development and survival of certain nerve cells. FD affects many different parts of the body, including the eyes, skin, lungs, and gastrointestinal system. Common symptoms include difficulty swallowing, vomiting, reflux, respiratory problems, abnormal sweating, blood pressure fluctuations, and abnormal sensitivity to pain and temperature. There is no cure for FD, but treatment options aim to manage the symptoms and improve quality of life. Treatment may include medications to improve gastrointestinal function, reduce blood pressure fluctuations, and control pain, as well as physical therapy and other supportive measures.
Familial mediterranean fever, which is a genetic disorder that causes recurrent episodes of fever and inflammation.
Familial non syndromic congenital heart disease, which is a group of conditions that affect the heart and are present at birth. They are caused by genetic mutations that affect the development of the heart.
Familial rhabdomyosarcoma, which is a rare type of cancer that affects the skeletal muscle tissue. It is caused by mutations in certain genes that control cell growth and division. Symptoms may include a lump or swelling in the affected area, pain, and difficulty moving.
Familial thoracic aortic aneurysm and aortic dissection, which is a disease that can lead to life-threatening complications. A genetic test may be used to assess an individual's risk for developing aortic aneurysms or dissections.
Febrile seizures, which are are seizures that can occur in children when they have a fever and can cause convulsions, loss of consciousness, and other symptoms.
Foot drop (foot dorsiflexor weakness), which is a medical condition characterized by the inability to lift the front part of the foot, resulting in a dragging or slapping gait.
Frontotemporal dementia, which is a type of dementia that affects the frontal and temporal lobes of the brain, leading to changes in behavior, language, and personality.
Gastric (stomach) cancer, which is a type of cancer that arises from the lining of the stomach. It can cause symptoms such as abdominal pain, nausea, and vomiting.
Gastrointestinal (GI) tract tumors, which are a type of cancer that can occur in any part of the digestive system, including the esophagus, stomach, small intestine, colon, rectum, and anus. GI tract tumors can be either benign or malignant, with malignant tumors being more common and potentially life-threatening. Symptoms of GI tract tumors can vary depending on the location and size of the tumor, but may include abdominal pain, nausea, vomiting, bloating, constipation or diarrhea, blood in the stool, and unintentional weight loss. Risk factors for GI tract tumors include age, family history, certain genetic syndromes, a diet high in red and processed meat, and a history of inflammatory bowel disease. Treatment options for GI tract tumors may include surgery, radiation therapy, chemotherapy, or a combination of these therapies, depending on the stage and location of the tumor.
Glycogen Storage disease II, which is a rare genetic disorder that affects the breakdown of glycogen in the body, leading to muscle weakness and fatigue.
Gout, which refers to a type of arthritis caused by the buildup of uric acid crystals in the joints. Genetic factors can play a role in an individual's risk of developing gout.
Granulomatosis, which is a group of rare autoimmune diseases that cause inflammation and damage to various organs and tissues.
Granulomatosis with Polyangitis (GPA), which is a rare autoimmune disease that causes inflammation in the blood vessels, leading to a range of symptoms including sinusitis, lung and kidney damage, and skin lesions.
Head and neck cancer susceptibility, which refers to an increased risk of developing tumors or cancers that originate in the tissues of the head and neck, including the mouth, throat, nose, and sinuses. Risk factors for head and neck cancer include tobacco and alcohol use, human papillomavirus (HPV) infection, exposure to certain chemicals or environmental factors, and family history of cancer. Symptoms of head and neck cancer may vary depending on the location and stage of the tumor, but may include mouth sores, difficulty swallowing, persistent hoarseness or sore throat, ear pain, and changes in voice or vision. Diagnosis of head and neck cancer is typically made through a combination of clinical exams, imaging tests, and biopsy of suspicious tissue. Treatment for head and neck cancer depends on the type, location, and stage of the tumor, as well as the individual's overall health. Options may include surgery, radiation therapy, chemotherapy, or a combination of these treatments. Prognosis for head and neck cancer varies depending on the type and stage of the cancer, as well as the effectiveness of treatment. Early detection and prompt treatment are important for improving outcomes and quality of life for affected individuals.
Headache, which refers to a condition where an individual experiences pain or discomfort in the head, scalp, or neck region. There are various causes of headaches, including genetic factors.
Hemifacial spasm, which is a condition characterized by involuntary muscle contractions on one side of the face, which can lead to twitching and facial distortion.
Hemiplegia, which is a condition in which one side of the body is paralyzed or has reduced movement due to damage in the brain.
Hepatocellular carcinoma (HCC), also known as liver cancer, which is a type of cancer that forms in the cells of the liver. It is typically associated with liver damage or disease, such as chronic hepatitis B or C infection, cirrhosis, or alcohol abuse. HCC can grow and spread quickly and may not cause symptoms until later stages of the disease. Symptoms of HCC may include abdominal pain, swelling, or tenderness, unexplained weight loss, and yellowing of the skin or eyes. Diagnosis of HCC is typically made through a combination of clinical exams, imaging tests, and biopsy of suspicious tissue. Treatment for HCC depends on the stage and severity of the cancer, but may include surgery, radiation therapy, chemotherapy, ablation therapy, or a combination of these treatments. The outlook for individuals with HCC varies depending on the stage and severity of the cancer, as well as the effectiveness of treatment. Early detection and prompt treatment are important for improving outcomes and quality of life for affected individuals.
Hereditary erythrocytosis, which is a rare genetic disorder that causes the production of too many red blood cells, leading to thickening of the blood and an increased risk of blood clots.
Hereditary neuropathy, which may be identified by using a genetic panel that tests for mutations associated with inherited forms of peripheral neuropathy.
Hereditary spastic paraplegia-adult onset, which is a group of inherited neurological disorders that primarily affect the legs and cause progressive stiffness and spasticity.
Hereditary spastic paraplegia, which is a group of genetic disorders that affect the nerves responsible for controlling movement in the legs, causing spasticity and weakness.
Langerhans cell histiocytosis (LCH), which is a rare disorder in which an excess of Langerhans cells, a type of white blood cell, build up in various parts of the body, causing damage to tissues and organs. LCH can affect people of any age, but it is most commonly diagnosed in children. Symptoms of LCH can vary depending on the location and extent of the disease, and may include bone pain, skin rash, fever, weight loss, and organ dysfunction. Treatment for LCH may involve chemotherapy, radiation therapy, and/or surgery depending on the severity of the disease. With appropriate treatment, the prognosis for LCH can be good, especially for those with limited involvement of the disease.
Hodgkin lymphoma, also known as Hodgkin's disease, which is a type of cancer that originates in white blood cells called lymphocytes. It typically starts in the lymph nodes and can spread to other parts of the body, including the spleen, liver, bone marrow, and lungs. Hodgkin lymphoma is characterized by the presence of Reed-Sternberg cells, which are large, abnormal cells that can be seen under a microscope. The exact cause of Hodgkin lymphoma is unknown, but it is believed to be related to genetic and environmental factors. Symptoms may include swollen lymph nodes, fever, night sweats, weight loss, and fatigue. Treatment for Hodgkin lymphoma typically involves chemotherapy, radiation therapy, or a combination of both. In some cases, stem cell transplantation may also be recommended. Prognosis and survival rates vary depending on the stage and grade of the cancer, as well as the patient's age, overall health, and response to treatment.
Holoprosencephaly, which is a rare birth defect that affects the development of the brain and face, resulting in a range of physical and cognitive abnormalities. The severity can vary widely, from mild facial abnormalities to severe brain malformations.
Huntington's disease, which is a genetic disorder that affects the brain and nervous system, causing movement problems, cognitive decline, and psychiatric symptoms.
Hydranencephaly, which is a rare neurological condition in which the cerebral hemispheres of the brain are largely or completely absent, causing severe developmental delay and intellectual disability.
Hereditary spastic paraplegia-childhood onset, which is a group of inherited disorders that affect the nerves responsible for controlling muscle movement in the legs, leading to progressive muscle stiffness and weakness.
Hydrocephalus, which is a condition where there is an accumulation of cerebrospinal fluid in the brain, which can lead to pressure on the brain and enlargement of the head. Symptoms may include headache, nausea, and vomiting.
Hyperlipidemia, also known as high blood cholesterol, which is a condition in which there are high levels of fats, such as cholesterol and triglycerides, in the blood. It is a risk factor for heart disease and stroke.
Hypersomnia, which is a sleep disorder characterized by excessive daytime sleepiness and prolonged nighttime sleep.
Hypertension or high blood pressure, which is a common condition in which the force of blood against the walls of the arteries is consistently too high. This can cause damage to the blood vessels and increase the risk of serious health problems such as heart disease, stroke, and kidney disease. Hypertension is often called the “silent killer” because it may not cause noticeable symptoms, but it can be diagnosed through routine blood pressure measurements. Risk factors for hypertension include age, family history, obesity, physical inactivity, smoking, and certain medical conditions such as diabetes and kidney disease. Treatment for hypertension may include lifestyle modifications such as weight loss, exercise, and dietary changes, as well as medications to lower blood pressure. Managing hypertension is important for reducing the risk of complications and maintaining overall health.
Hypertonia, which is a condition characterized by increased muscle tone or stiffness, which can cause difficulty with movement and coordination. Hypertonia can be caused by a variety of factors, including neurological disorders such as cerebral palsy, stroke, or Parkinson's disease, as well as muscle and joint disorders. Symptoms of hypertonia may include muscle rigidity, spasticity (involuntary muscle contractions), and difficulty with fine motor tasks such as writing or buttoning clothes. Treatment for hypertonia may include physical therapy, which can help improve muscle strength and range of motion, as well as medications such as muscle relaxants to reduce muscle stiffness and spasticity. In severe cases, surgical interventions such as tendon lengthening or nerve stimulation may be considered.
Hypertrophic cardiomyopathy (HCM), which is a genetic condition in which the heart muscle becomes abnormally thick, making it harder for the heart to pump blood effectively. HCM can affect people of any age, and it is a leading cause of sudden cardiac death in young people, particularly athletes. Symptoms of HCM may include shortness of breath, chest pain, fainting, and palpitations. HCM is caused by mutations in genes that regulate the structure and function of heart muscle cells, and it is typically diagnosed through a combination of physical exams, imaging tests such as echocardiography, and genetic testing. Treatment for HCM may include medications to control symptoms, lifestyle modifications such as avoiding strenuous exercise, and surgery or implantation of a device to manage complications such as abnormal heart rhythms. Regular monitoring and management of HCM are important for preventing complications and improving outcomes.
Hypertrophic cardiomyopathy (HCM), which is a genetic heart disease that affects the structure and function of the heart muscle. It is characterized by thickening of the heart muscle, particularly in the left ventricle, which can lead to arrhythmias, heart failure, and sudden cardiac arrest. HCM can occur at any age, but it is often diagnosed in teenagers and young adults. Symptoms of HCM may include chest pain, shortness of breath, palpitations, fatigue, and dizziness. Diagnosis of HCM is usually made through a combination of clinical exams, imaging tests, and genetic testing. Treatment for HCM focuses on managing symptoms and preventing complications, and may include medications, surgical interventions such as septal myectomy or alcohol septal ablation, and implantable devices such as pacemakers or defibrillators. The prognosis for individuals with HCM varies depending on the severity of the condition and the effectiveness of treatment. Regular monitoring and management of HCM are important for improving outcomes and preventing complications such as sudden cardiac arrest.
Hypotonia, which is a medical condition characterized by decreased muscle tone or a lack of muscle tension, resulting in reduced or weak muscle strength and poor posture. It can affect people of all ages and can be caused by a variety of factors, such as neurological disorders, genetic conditions, or metabolic disorders. Symptoms of hypotonia may include poor muscle tone, difficulty with movement and coordination, delayed developmental milestones, and difficulty with speech and swallowing. Diagnosis of hypotonia is usually made through a combination of clinical exams, genetic testing, and imaging tests. Treatment for hypotonia depends on the underlying cause and severity of the condition, but may include physical therapy, occupational therapy, speech therapy, and other forms of rehabilitation to improve muscle strength and coordination. In some cases, medication or surgery may also be considered. Early detection and management of hypotonia are important for preventing complications and improving quality of life for affected individuals and their families.
Idiopathic pulmonary fibrosis (IPF), which is a chronic lung disease that causes scarring of the lungs, leading to difficulty breathing and other symptoms.
Incontinentia pigmenti (IP), which is a rare genetic disorder that primarily affects the skin, hair, teeth, and nervous system. It is caused by mutations in the IKBKG gene, which provides instructions for making a protein called NEMO. IP is typically diagnosed in infancy or early childhood and affects females more frequently than males. Symptoms of IP may include skin rashes, blisters, and pigmentation changes, dental abnormalities, hair loss or thinning, and neurological abnormalities such as seizures or developmental delays. Diagnosis of IP is typically made through a combination of clinical exams, imaging tests, and genetic testing. Treatment for IP may involve managing symptoms such as skin rashes or infections, and addressing complications such as seizures or developmental delays. The prognosis for individuals with IP varies depending on the severity of the condition, the presence of associated complications, and the effectiveness of treatment. Early detection and management of IP are important for improving outcomes and quality of life for affected individuals and their families.
Infantile Refsum disease, which is a rare genetic disorder characterized by the accumulation of a fatty acid called phytanic acid in the body. This accumulation leads to a range of symptoms, including muscle weakness, vision and hearing loss, developmental delay, and intellectual disability. Other features of the condition can include abnormal facial features, skeletal abnormalities, and liver problems. Infantile Refsum disease is caused by mutations in the PEX1 or PEX6 genes, which are involved in the formation of peroxisomes, cellular structures involved in the breakdown of phytanic acid. There is no cure for Infantile Refsum disease, and treatment is based on managing symptoms and supporting overall health and development. This may include dietary modifications to limit the intake of phytanic acid, medications to manage symptoms, and therapies such as physical and occupational therapy.
Infantile spasms, which is a type of epilepsy that affects infants and young children, causing seizures that involve sudden, jerking movements of the arms and legs.
Inherited non-medullary thyroid cancer, which is a rare form of thyroid cancer that is passed down through families and is not associated with other thyroid disorders.
Inherited pancreatic cancer, which is a genetic testing panel that analyzes genes associated with an increased risk of developing pancreatic cancer.
Inherited predisposition to acute myeloid leukemia (AML), which may be determined using a genetic test with assessment of an individual's risk of developing acute myeloid leukemia, based on specific genetic mutations that are associated with an increased risk. Inherited renal cancer, which is a genetic panel that tests for mutations associated with inherited forms of kidney cancer.
Inherited white matter disorders (leukodystrophy) (v1.130): A group of rare genetic disorders that affect the white matter of the brain, causing a variety of neurological symptoms. Intellectual disability, which is a condition characterized by significant limitations in cognitive and adaptive functioning, typically with an onset in childhood. It may be caused by genetic, environmental, or unknown factors.
Iron deficiency anemia, which is a condition where there is a decrease in the number of red blood cells due to a lack of iron. Symptoms may include fatigue, weakness, and shortness of breath.
Kennedy's disease, which is a rare genetic disorder that affects the muscles and nerves, causing weakness and wasting of the limbs and sometimes respiratory and swallowing difficulties.
Krabbe disease, which is a rare genetic disorder that affects the nervous system, causing developmental delays, muscle weakness, and vision and hearing loss.
Laryngeal cancer, which is a type of cancer that affects the larynx (voice box).
Leukemia, which is a type of cancer that affects the blood cells, causing abnormal growth and proliferation of white blood cells.
Limb girdle muscular dystrophy, which is an inherited disorder that affects the muscles of the hips and shoulders, causing muscle weakness and wasting.
Lipoid proteinosis, which is a rare genetic disorder that affects the skin and other parts of the body, causing hoarseness, skin lesions, and other symptoms.
Lissencephaly, which is a rare brain malformation that can cause developmental delays, seizures, and other neurological problems.
Long QT syndrome (LQTS), which is a genetic disorder that affects the electrical system of the heart, resulting in abnormal heart rhythms that can lead to fainting or sudden cardiac arrest. The condition is characterized by a prolonged QT interval on an electrocardiogram (ECG). Symptoms may include palpitations, dizziness, or sudden loss of consciousness. LOTS can be caused by mutations in any of several genes that regulate the flow of ions in and out of heart cells. Treatment for LQTS may include medications to control heart rhythms, beta-blockers to reduce the risk of arrhythmias, or an implantable cardioverter defibrillator (ICD) to monitor the heart and deliver an electrical shock if needed. Individuals with LOTS should avoid triggers that may increase the risk of arrhythmias, such as certain medications or intense physical activity. Regular monitoring by a cardiologist is also recommended to manage the condition and prevent complications.
Lung cancer, which is a type of cancer that begins in the lungs and can spread to other parts of the body.
Lymphoma, which is a type of cancer that affects the lymphatic system, which is part of the body's immune system. Symptoms can include enlarged lymph nodes, fever, night sweats, and weight loss.
Medulloblastoma, which is a type of brain tumor that mostly affects children and can cause headaches, vomiting, and difficulty with movement or balance.
Megalencephaly, which is a condition in which the brain is abnormally large, causing developmental delay, intellectual disability, seizures, and other neurological symptoms.
Melanoma pertinent cancer susceptibility, which may be determined using a genetic test that looks for mutations in genes that increase the risk of developing melanoma, a type of skin cancer.
Meningitis, which is an inflammation of the protective membranes that cover the brain and spinal cord. It can be caused by bacterial, viral, or fungal infections and can be life-threatening.
Menkes disease, which is a rare genetic disorder that affects copper metabolism and leads to a copper deficiency in the body. It can cause developmental delays, seizures, and other neurological symptoms.
Microcephaly, which is a neurological condition characterized by an abnormally small head size and underdeveloped brain. This can result in a range of symptoms, including developmental delays, intellectual disability, seizures, and difficulty with movement and balance. Microcephaly can be caused by a variety of factors, including genetic mutations, infections during pregnancy (such as Zika virus), exposure to toxins, and malnutrition. The severity of symptoms can vary widely, from mild to severe, and there is currently no cure for Microcephaly. Treatment options may include physical therapy, occupational therapy, and speech therapy, as well as medications to manage seizures or other associated symptoms.
Migraine, which is a neurological condition characterized by recurring headaches that can range from moderate to severe in intensity. Migraine headaches are often accompanied by other symptoms such as nausea, vomiting, and sensitivity to light and sound. Migraine can be triggered by a variety of factors, including stress, certain foods or drinks, hormonal changes, and changes in sleep patterns. Diagnosis of migraine is usually made based on the individual's symptoms and medical history, and other conditions that may cause similar symptoms are typically ruled out. Treatment for migraine may include medication, lifestyle modifications, and alternative therapies such as acupuncture or biofeedback. The goal of treatment is to manage symptoms and prevent future attacks. Prognosis for migraine varies depending on the individual, the severity of the condition, and the effectiveness of treatment. With proper management, many individuals with migraine are able to lead healthy and productive lives.
Mitochondrial myopathy, which is a type of genetic neuromuscular disorder that affects the mitochondria, which are responsible for producing energy in cells. The condition can cause muscle weakness, exercise intolerance, and fatigue, as well as other symptoms such as vision and hearing problems, gastrointestinal issues, and difficulty swallowing. Symptoms may appear at any age and can vary widely in severity. There is no cure for mitochondrial myopathy, but treatment can help manage symptoms and improve quality of life. This may include physical therapy, exercise programs, and medications to manage specific symptoms, such as coenzyme Q10 for fatigue and antioxidants to reduce oxidative stress.
Monomelic amyotrophy (MMA), also known as Hirayama disease, which is a rare neurological disorder that affects young adults, most commonly males. It is characterized by progressive weakness and wasting of the muscles in one or more limbs, typically affecting the hand, forearm, and sometimes the shoulder. The cause of MMA is not well understood, but it is thought to be related to nerve compression or damage in the spinal cord. Symptoms of MMA may include muscle weakness, atrophy, and twitching, as well as cold intolerance and numbness in the affected limb. There is no cure for MMA, but treatment options include physical therapy, orthotic devices, and surgery to release nerve compression. With appropriate treatment, many people with MMA can maintain function and lead a normal life.
Moyamoya Disease, which is a rare, progressive disorder that affects the blood vessels in the brain, causing narrowing or blockage and increasing the risk of stroke.
Mucopolysaccharideosis, Gaucher, Fabry (v1.2): A group of inherited metabolic disorders that affect the body's ability to break down certain sugars and lipids, leading to a variety of symptoms.
Multiple endocrine tumors, which is a genetic disorder that predisposes individuals to developing tumors in multiple endocrine organs, such as the thyroid, parathyroid, adrenal gland, and pancreas.
Multiple myeloma/plasma cell neoplasms, which are a group of cancers that develop in plasma cells, a type of white blood cell that produces antibodies. Symptoms include bone pain, fatigue, and recurrent infections.
Multiple system atrophy, which is a rare neurological disorder that affects multiple parts of the brain and nervous system, leading to movement and autonomic dysfunction.
Multiple system atrophy with orthostatic hypotension, which is a rare neurodegenerative disorder that affects the autonomic nervous system, causing a range of symptoms including orthostatic hypotension (a sudden drop in blood pressure upon standing).
Muscular dystrophy, which is a group of genetic disorders that cause progressive weakness and degeneration of muscles.
Myelodysplasia, which is a group of blood disorders in which the bone marrow does not produce enough healthy blood cells, which can lead to anemia and other complications.
Chronic myelogenous leukemia (CML), which is a type of cancer that affects the blood and bone marrow. It is characterized by the presence of an abnormal chromosome called the Philadelphia chromosome. Symptoms may include fatigue, weakness, and anemia.
Myocardial infarction, commonly known as a heart attack, which occurs when blood flow to a part of the heart is blocked, typically by a blood clot. The lack of blood flow and oxygen to the heart muscle can cause damage and lead to chest pain or discomfort, shortness of breath, fatigue, and other symptoms. Risk factors for heart attack include age, family history, smoking, high blood pressure, high cholesterol, obesity, and diabetes. Treatment for heart attack typically involves emergency medical care to restore blood flow to the heart, which may include medications to dissolve the clot or angioplasty to open the blocked artery. After treatment, lifestyle changes such as regular exercise, a healthy diet, and medication may be recommended to manage risk factors and prevent future heart problems.
Myoclonus, which is a neurological disorder characterized by sudden, involuntary muscle twitches or jerks. It may be caused by a variety of underlying conditions, including genetic mutations, metabolic disorders, or brain injuries.
Myopathy, which is a condition in which there is weakness or wasting of muscles, and it can be caused by various genetic or acquired factors.
Narcolepsy, which is a neurological disorder that affects sleep-wake cycles. It is characterized by excessive daytime sleepiness, sleep paralysis, and vivid hallucinations.
Neoplasm, which is a general term that refers to an abnormal growth of cells that can be either benign (not cancerous) or malignant (cancerous). Neoplasms can occur in any part of the body and can vary widely in size, shape, and appearance. Benign neoplasms do not spread to other parts of the body and are not usually life-threatening, while malignant neoplasms can invade nearby tissues and spread to other parts of the body, potentially causing serious health problems. Treatment for neoplasms depends on the type, location, and stage of the growth, and may include surgery, radiation therapy, chemotherapy, or a combination of these treatments. Early detection and prompt treatment are important for improving the chances of successful treatment and long-term survival.
A neoplasm of the heart, which refers to an abnormal growth or tumor that develops in the heart. These tumors can be benign (non-cancerous) or malignant (cancerous). Primary tumors of the heart, which originate in the heart tissue, are rare, accounting for less than 5% of all cardiac tumors. Secondary tumors, which have spread to the heart from other parts of the body, are more common. Symptoms of cardiac tumors may include chest pain, shortness of breath, irregular heartbeat, fatigue, and swelling in the legs and abdomen. The diagnosis of cardiac tumors is typically made through imaging tests such as echocardiography, CT scans, and MRI scans. Treatment for cardiac tumors depends on the type, location, and stage of the tumor, as well as the overall health of the individual. Options may include surgery to remove the tumor, radiation therapy, chemotherapy, or a combination of these treatments. Prognosis for cardiac tumors varies widely, depending on the type and stage of the tumor, and the effectiveness of treatment. Early detection and prompt treatment are important for improving outcomes and preventing complications such as heart failure or stroke.
Nephroblastoma, also known as Wilms' tumor, which is a rare type of childhood kidney cancer that typically affects children under the age of five. It is characterized by the presence of abnormal cells in one or both kidneys that can grow and spread quickly to other parts of the body. Symptoms of nephroblastoma may include abdominal swelling, pain, or tenderness, fever, and blood in the urine. Diagnosis of nephroblastoma is typically made through a combination of clinical exams, imaging tests, and biopsy of suspicious tissue. Treatment for nephroblastoma usually involves surgery to remove the tumor, as well as chemotherapy and/or radiation therapy to destroy any remaining cancer cells. The outlook for children with nephroblastoma varies depending on the stage and severity of the cancer, as well as the effectiveness of treatment. Early detection and prompt treatment are important for improving outcomes and quality of life for affected children and their families.
Neuroblastoma, which is a type of cancer that develops in immature nerve cells, most commonly found in the adrenal glands, but can also occur in the chest, neck, or pelvis. It is the most common type of cancer in infants and young children. Symptoms may include abdominal swelling, pain, fever, weight loss, and fatigue. Treatment for neuroblastoma may include surgery, chemotherapy, radiation therapy, and immunotherapy, depending on the stage and location of the cancer. The prognosis for neuroblastoma can vary widely depending on various factors, including age at diagnosis, tumor size, and whether the cancer has spread to other parts of the body.
Neurodegeneration with brain iron accumulation 2A (NBIA2A), which is a rare genetic disorder that affects the nervous system. It is characterized by an abnormal buildup of iron in the brain, which leads to progressive degeneration of the nervous system. Symptoms typically begin in childhood and may include movement disorders, muscle rigidity, dystonia, spasticity, ataxia, and cognitive impairment. The disorder is caused by mutations in the PLA2G6 gene, which provides instructions for making an enzyme involved in the breakdown of cellular membranes. There is no cure for NBIA2A, and treatment is largely symptomatic and supportive, with a focus on managing symptoms such as muscle rigidity and movement disorders.
Neuroendocrine tumors (NETs), which is a rare type of cancer that originate in specialized cells called neuroendocrine cells, which are found throughout the body. Neuroendocrine cancer susceptibility refers to genetic mutations that increase an individual's risk of developing NETs. These mutations can be inherited or occur spontaneously. Some of the genes that have been associated with an increased risk of developing NETs include the MEN1, RET, and SDHB genes. Individuals with a family history of NETs, multiple endocrine neoplasia type 1 (MEN1), or other related conditions may be advised to undergo genetic testing to assess their risk. Treatment for NETs depends on the location, stage, and grade of the tumor, and may include surgery, radiation therapy, chemotherapy, or targeted therapy.
Neurofibromatosis Type 1, which is a genetic disorder that causes the growth of non-cancerous tumors in the nervous system, skin, and other organs. It can cause a range of symptoms, including skin pigmentation, bone deformities, and vision problems.
Neuroma, which is a type of benign tumor that develops on the nerve tissue, causing pain and other symptoms.
Non-CF bronchiectasis, which is a chronic lung disease in which the bronchi, the airways that carry air to the lungs, become damaged and widened. This damage can cause mucus to build up and create an environment for bacterial growth, leading to recurrent lung infections and breathing difficulties. Non-CF bronchiectasis can be caused by a variety of factors, including infections, immune system disorders, and structural abnormalities of the airways. Symptoms of the condition may include chronic cough, sputum production, shortness of breath, fatigue, and recurrent respiratory infections. Treatment for non-CF bronchiectasis often involves medication to control symptoms and prevent infection, as well as airway clearance techniques and pulmonary rehabilitation to improve lung function and quality of life.
Noncompaction cardiomyopathy (NCC), which is a rare genetic heart condition that affects the muscle tissue of the heart, causing it to develop a sponge-like appearance with deep grooves or trabeculations. This can lead to abnormal heart rhythms, heart failure, and blood clotting, which can increase the risk of stroke. Symptoms may include fatigue, shortness of breath, chest pain, and palpitations. Treatment for NCC may involve medications to manage symptoms, as well as surgical procedures or implantable devices to correct abnormal heart rhythms or improve heart function.
Non-Hodgkin Lymphoma, which is a type of cancer that affects the lymphatic system, which is a part of the immune system that helps fight infection and disease.
Normal pressure hydrocephalus (NPH), which is a neurological disorder characterized by an abnormal buildup of cerebrospinal fluid (CSF) in the brain's ventricles. NPH typically affects older adults, and its symptoms can be similar to those of other neurological disorders, such as Alzheimer's and Parkinson's disease. Symptoms of NPH may include gait disturbances (difficulty walking), urinary incontinence, and cognitive impairment. The diagnosis of NPH is made through imaging tests such as MRI and CT scans, and a lumbar puncture to measure the pressure of the CSF. Treatment for NPH may involve surgical insertion of a shunt to drain excess fluid from the brain, which can improve symptoms in many cases. However, not all cases of NPH are amenable to shunting, and treatment outcomes can vary widely depending on the individual and other factors. Early detection and treatment of NPH are important for improving outcomes and quality of life for affected individuals.
Olivopontocerebellar atrophy (OPCA), which is a rare progressive neurological disorder that affects the brainstem and cerebellum, which control balance, coordination, and other motor functions. It is caused by the degeneration of specific neurons in these regions, leading to symptoms such as unsteady gait, tremors, and difficulty with speech and swallowing. Other symptoms may include muscle stiffness, weakness, and vision problems. OPCA can be inherited or develop sporadically. Treatment aims to manage symptoms, and may involve physical therapy, medications to improve motor function, and speech therapy or feeding tubes to address swallowing difficulties.
Opsoclonus-myoclonus syndrome (OMS), which is a rare neurological disorder characterized by uncontrolled eye movements (opsoclonus) and involuntary muscle jerks (myoclonus). The cause of OMS is not well understood, but it often occurs in association with tumors, infections, or autoimmune disorders. In children, it is commonly associated with neuroblastoma, a type of cancer that develops from nerve cells. Symptoms of OMS may also include ataxia (difficulty with coordination and balance), irritability, and sleep disturbances. Treatment options include corticosteroids, immunotherapy, chemotherapy, and surgery, depending on the underlying cause of the disorder. Early diagnosis and treatment are important to prevent complications and improve outcomes.
Orthostatic Hypotension, which is a condition in which a person experiences a sudden drop in blood pressure when standing up, leading to symptoms such as dizziness, lightheadedness, and fainting.
Osteoarthritis, which is a type of arthritis that occurs when the cartilage in joints breaks down, causing pain and stiffness.
Osteoporosis, which is a medical condition characterized by weakened bones that are more likely to fracture. It is most commonly seen in older adults and postmenopausal women. Osteosarcoma (bone cancer), which is a type of cancer that develops in the bones, often affecting children and young adults.
Ovarian cancer pertinent cancer susceptibility, which may be determined using a genetic test to assess an individual's risk of developing ovarian cancer based on specific genetic mutations that are associated with an increased risk.
Pain, which is an unpleasant sensory and emotional experience associated with actual or potential tissue damage, or described in terms of such damage.
Paraganglioma, which is a rare tumor that arises from cells in the nervous system and can cause high blood pressure and other symptoms.
Parathyroid cancer, which is a rare cancer that develops in the parathyroid glands, which are responsible for regulating calcium levels in the body.
Paresthesia, which is a condition that causes numbness, tingling, or burning sensations in the body.
Parkinson disease and complex parkinsonism, which may be determined using a genetic test to assess an individual's risk of developing Parkinson's disease and related conditions based on specific genetic mutations that are associated with an increased risk.
Paroxysmal choreoathetosis, which is a rare neurological disorder that causes episodes of involuntary movements and abnormal postures.
Periodic Fever, Aphthous Stomatitis, Pharyngitis, Adenitis Syndrome (PFAPA) is a condition that typically affects children and is characterized by recurrent episodes of fever, mouth sores, sore throat, and swollen glands.
Periodic Paralysis, which is a rare condition characterized by episodes of muscle weakness or paralysis that may last for hours or days. It can be inherited or acquired, and is caused by changes in ion channels that regulate the electrical activity of muscles.
Peripheral neuropathy, which is a condition characterized by damage to the peripheral nerves, which can cause numbness, tingling, and weakness in the limbs.
Periventricular leukomalacia, which is a brain injury that occurs in premature infants, leading to motor and cognitive impairments.
Personality disorder/bipolar disorder (BPD), which is a mental health condition characterized by mood swings, impulsivity, and difficulty regulating emotions.
Pheochromocytoma, which is a rare tumor of the adrenal gland that produces excess adrenaline and other hormones, leading to symptoms such as high blood pressure and rapid heartbeat.
Pleuritis, which is inflammation of the lining of the lungs and chest cavity that can cause chest pain, coughing, and difficulty breathing.
Pleuropulmonary blastoma (lung cancer), which is a rare type of lung cancer that typically affects young children and is characterized by the growth of abnormal cells in the lungs and pleura.
Pneumonia, which is a lung infection that can be caused by various bacteria, viruses, and fungi. Symptoms include cough, fever, and difficulty breathing.
Polycythemia, which is a condition in which there is an increased number of red blood cells, which can cause blood clots and other complications.
Primary ciliary disorders (PCDs), which is a group of genetic conditions that affect the function of cilia, hair-like structures that line the respiratory tract, reproductive system, and other parts of the body. In PCDs, cilia do not work properly, leading to recurrent respiratory infections, chronic cough, and impaired fertility. Other symptoms may include hearing loss, nasal congestion, and recurrent ear infections. PCDs are caused by mutations in various genes that are involved in cilia structure and function. There is no cure for PCDs, and treatment options are mainly aimed at managing the symptoms and preventing complications. This may include antibiotics to treat infections, chest physiotherapy to clear mucus from the lungs, and surgery to address structural abnormalities in the respiratory tract or the reproductive system. Early diagnosis and management of PCDs are important for preventing long-term complications such as lung damage and infertility.
Primary Ciliary Dyskinesia (PCD), which is a rare genetic disorder that affects the function of cilia, which are hair-like structures that line the respiratory tract, the reproductive system, and the brain ventricles. In PCD, the cilia do not work properly, leading to recurrent respiratory infections, chronic cough, and impaired fertility. Other symptoms may include hearing loss, nasal congestion, and recurrent ear infections. PCD is caused by mutations in various genes that are involved in cilia structure and function. There is no cure for PCD, and treatment options are mainly aimed at managing the symptoms and preventing complications. This may include antibiotics to treat infections, chest physiotherapy to clear mucus from the lungs, and surgery to address structural abnormalities in the respiratory tract or the reproductive system. Early diagnosis and management of PCD are important for preventing long-term complications such as lung damage and infertility.
Primary Lateral Sclerosis, which is a rare neurological disorder that affects the upper motor neurons, leading to muscle weakness and spasticity in the limbs. It is a progressive condition with no known cure.
Progressive cardiac conduction disease (PCCD), which is an inherited condition that affects the electrical system of the heart, leading to abnormal heart rhythms and other symptoms.
Prostate cancer, which is a type of cancer that develops in the prostate gland, which is part of the male reproductive system. Symptoms can include difficulty urinating, blood in the urine or semen, and pain in the hips, back, or chest. A group of genetic mutations may increase the risk of developing prostate cancer.
Pulmonary embolism, which is a condition where a blood clot forms in the deep veins of the legs or pelvis and travels to the lungs, causing a blockage in the pulmonary arteries. Symptoms may include sudden onset of shortness of breath, chest pain, coughing up blood, and rapid heartbeat.
Purpura, which is a condition characterized by the appearance of small purple or red spots on the skin and mucous membranes, caused by bleeding under the skin.
Rare anemia, which is a genetic test that assesses an individual's risk for inherited forms of anemia that are caused by mutations in specific genes
Rare multisystem ciliopathy disorders, which is a genetic testing panel that analyzes genes associated with a group of rare disorders that affect the function of tiny hair-like structures called cilia in various organs and tissues.
Raynaud's phenomenon, which is a condition that affects the blood vessels, causing them to narrow and reduce blood flow to the extremities, usually the fingers and toes, but can also affect the nose, ears, and lips. This can result in coldness, numbness, tingling, or a painful sensation in the affected areas, and the skin may turn white or blue before becoming red as the blood vessels open up again. Raynaud's Phenomenon can be triggered by cold temperatures, stress, or certain medications. In some cases, it can occur on its own (primary Raynaud's), while in other cases, it may be associated with an underlying condition such as autoimmune diseases, connective tissue disorders, or occupational exposure to certain chemicals (secondary Raynaud's). Treatment options for Raynaud's Phenomenon may include lifestyle modifications such as avoiding triggers, keeping the extremities warm, and quitting smoking, as well as medications to improve blood flow. Refsum disease, which is a rare genetic disorder that can cause vision and hearing problems, muscle weakness, and other symptoms.
Renal cancer pertinent cancer susceptibility, which may be determined using a genetic test that assesses an individual's risk for developing kidney cancer based on inherited gene mutations that are associated with this type of cancer.
Respiratory ciliopathies including non-CF bronchiectasis, which is a group of rare genetic disorders that affect the function of the cilia, hair-like structures that line the respiratory tract and help to clear mucus and other substances from the lungs.
Retinoblastoma, which is a type of eye cancer that usually affects children and can cause vision problems, eye pain, and other symptoms.
Rett Syndrome, which is a rare genetic disorder that primarily affects girls, causing developmental delay, intellectual disability, and loss of motor skills.
Rhabdoid tumor predisposition, which is a genetic disorder that increases the risk of developing rhabdoid tumors, which are rare but aggressive cancers that can occur in various parts of the body.
Rhabdomyosarcoma (soft tissue sarcoma), which is a type of cancer that develops in the skeletal muscle or connective tissue, most commonly seen in children and young adults.
Sandhoff disease, which is a rare genetic disorder that affects the nervous system. It is caused by a deficiency in an enzyme that breaks down a type of fat called gangliosides. Symptoms may include progressive muscle weakness, seizures, and developmental delays.
Sarcoidosis, which is a condition where clusters of inflammatory cells (granulomas) form in various organs of the body, most commonly the lungs, lymph nodes, and skin. It can cause a range of symptoms, including fatigue, shortness of breath, and skin rashes.
Sarcoma, which is a type of cancer that arises from the connective tissues, such as bone, cartilage, fat, muscle, or blood vessels. Sarcomas are rare and account for only about 1% of all cancers. While the exact cause of sarcoma is unknown, there are certain factors that may increase a person's susceptibility to developing the disease. These include genetic mutations, exposure to radiation, certain inherited syndromes, and certain viruses. Some inherited syndromes that increase the risk of sarcoma include Li-Fraumeni syndrome, neurofibromatosis type 1, and retinoblastoma. If you have a family history of sarcoma or an inherited syndrome that increases the risk of the disease, it is important to discuss your risk with a healthcare provider and consider appropriate screening and surveillance measures. Sarcoma susceptibility refers to an increased risk of developing sarcoma, which is a type of cancer that affects the connective tissues such as bones, muscles, cartilage, and blood vessels. Sarcoma susceptibility can be caused by various factors, including genetic mutations, exposure to radiation or certain chemicals, and previous treatment with chemotherapy or radiation therapy for other cancers. Some inherited genetic syndromes are also associated with an increased risk of developing sarcoma, such as Li-Fraumeni syndrome and neurofibromatosis. Symptoms of sarcoma may include pain, swelling, or a lump in the affected area, and the diagnosis is typically confirmed through biopsy and imaging tests. Treatment for sarcoma depends on the type, location, and stage of the cancer and may include surgery, radiation therapy, chemotherapy, or a combination of these treatments. Early detection and prompt treatment are important for improving the chances of successful treatment and long-term survival.
Schizencephaly, which is a rare birth defect characterized by abnormal clefts or gaps in the brain's cerebral hemispheres. It can lead to developmental delays, seizures, and other neurological symptoms.
Schizophrenia, which is a chronic mental disorder that affects how a person thinks, feels, and behaves. Symptoms can include hallucinations, delusions, disorganized speech, and a lack of motivation.
Scleroderma, which is a chronic autoimmune disease that affects the connective tissues of the body, leading to hardening and thickening of the skin and other organs. Symptoms may include skin changes, joint pain, and internal organ involvement.
Severe microcephaly, which is a condition in which the head circumference of an infant is significantly smaller than average for their age and sex, indicating an abnormally small brain. This can result in developmental delays, intellectual disability, and neurological problems. Severe microcephaly can be caused by genetic mutations, infections during pregnancy, exposure to toxins or radiation, or other factors that disrupt normal brain development. Infants with severe microcephaly may also have other physical abnormalities, such as seizures, hearing and vision problems, and feeding difficulties. There is no cure for severe microcephaly, and treatment options are mainly aimed at managing symptoms and providing supportive care. This may include physical therapy, speech therapy, and other interventions to promote developmental and functional abilities. Early diagnosis and management of severe microcephaly are important for providing the best possible outcomes and support to affected individuals and their families.
Sezary syndrome (lymphoma), which is a rare type of cutaneous T-cell lymphoma that affects the skin and lymph nodes. It is a type of non-Hodgkin lymphoma that is characterized by abnormal T-cells, a type of white blood cell, in the blood and skin. Sezary syndrome typically presents with skin lesions, generalized itching, and a rash that covers most of the body. It may also be associated with enlarged lymph nodes, fever, weight loss, and fatigue. The diagnosis of Sezary syndrome is made through a combination of clinical exams, skin biopsies, and blood tests. Treatment for Sezary syndrome may include chemotherapy, radiation therapy, and targeted therapies that aim to attack the abnormal T-cells. Other supportive measures such as antibiotics, antihistamines, and topical treatments may also be used to manage symptoms. Early detection and treatment of Sezary syndrome are important for improving outcomes and providing supportive care to affected individuals and their families.
Skeletal muscle channelopathy (v1.31) is a genetic disorder that affects the functioning of ion channels in skeletal muscle cells, resulting in muscle stiffness, weakness, and cramping.
Sleep Apnea, which is a common sleep disorder in which a person's breathing repeatedly stops and starts during sleep. The most common type of sleep apnea is Obstructive Sleep Apnea (OSA), which occurs when the muscles in the back of the throat fail to keep the airway open, leading to breathing interruptions. This can result in loud snoring, gasping or choking sounds during sleep, daytime fatigue, and difficulty concentrating. Risk factors for sleep apnea include obesity, older age, being male, having a family history of the condition, and certain medical conditions such as high blood pressure or diabetes. Treatment for sleep apnea may include lifestyle changes such as weight loss, avoiding alcohol and sedatives, and sleeping in a specific position, as well as continuous positive airway pressure (CPAP) therapy, which involves wearing a mask over the nose and/or mouth during sleep to keep the airway open.
Small cell lung cancer (SCLC), which is a type of lung cancer that typically grows and spreads quickly, often before symptoms appear. It is characterized by small cells that grow rapidly and can spread to other parts of the body, including the lymph nodes, liver, and bones. SCLC is strongly associated with smoking, and most cases occur in individuals who are current or former smokers. Symptoms of SCLC may include coughing, chest pain, shortness of breath, fatigue, and unintended weight loss. Diagnosis of SCLC is usually made through a combination of clinical exams, imaging tests, and biopsy of suspicious tissue. Treatment for SCLC depends on the stage and severity of the cancer, but may include surgery, radiation therapy, chemotherapy, or a combination of these treatments. Prognosis for SCLC varies depending on the stage and severity of the cancer, as well as the effectiveness of treatment. Early detection and prompt treatment are important for improving outcomes and quality of life for affected individuals.
Soft tissue sarcoma, which is a rare type of cancer that develops in the soft tissues of the body, including muscles, tendons, fat, and blood vessels. It can occur in any part of the body and can present as a painless lump or swelling. Treatment options for soft tissue sarcoma may include surgery to remove the tumor, radiation therapy, and chemotherapy, depending on the stage and location of the cancer. The prognosis for soft tissue sarcoma can vary depending on various factors, including the size and location of the tumor, the stage of the cancer, and the patient's overall health.
Sotos syndrome, which is a rare genetic disorder that affects growth and development during childhood. It is caused by mutations or deletions in the NSD1 gene, which plays a role in regulating cell growth and division. Symptoms of Sotos syndrome may include overgrowth of the body, head, and limbs, delayed developmental milestones, learning disabilities, and behavioral problems. Diagnosis of Sotos syndrome is typically made through a combination of clinical exams, genetic testing, and imaging tests. Treatment for Sotos syndrome focuses on managing symptoms and may include physical therapy, occupational therapy, speech therapy, and other forms of supportive care. The outlook for individuals with Sotos syndrome varies depending on the severity of the condition and the effectiveness of treatment. Early detection and management of Sotos syndrome are important for improving outcomes and providing supportive care to affected individuals and their families.
Spasticity, which is a condition in which certain muscles in the body become stiff and difficult to move, often causing involuntary muscle spasms or contractions. It can be caused by a variety of neurological conditions such as stroke, multiple sclerosis, cerebral palsy, or spinal cord injury. Spasticity may affect different parts of the body, including the arms, legs, hands, or feet, and can lead to difficulties with movement, coordination, and balance. Treatment for spasticity may involve medications to relax the muscles, physical therapy to improve strength and flexibility, or invasive treatments such as surgery or injections of botulinum toxin. The management of spasticity aims to improve quality of life and prevent complications such as pain, contractures, or deformities.
Spina bifida, which is a birth defect that occurs when the spinal column fails to develop properly during fetal development, resulting in incomplete closure of the vertebrae. The severity of the condition can vary widely, with some individuals having no symptoms, while others may experience significant nerve damage, paralysis, and other complications. The most common form of spina bifida is myelomeningocele, which involves a sac of nerves and spinal fluid that protrudes through the opening in the spine. Other forms include meningocele, which involves only the protective covering around the spinal cord protruding through the opening, and spina bifida occulta, which is a mild form of the condition that may not cause any symptoms. Treatment for spina bifida typically involves surgery to repair the defect and prevent further nerve damage, as well as ongoing medical management to address any complications that may arise. With appropriate treatment, many individuals with spina bifida can lead relatively normal lives, although the degree of disability can vary depending on the severity of the condition.
Spinal canal stenosis, which is a medical condition characterized by narrowing of the spinal canal, which can put pressure on the spinal cord and nerves. It can occur in any part of the spine but is most common in the lower back and neck. Spinal canal stenosis can be caused by a variety of factors, including degenerative changes due to aging, herniated discs, arthritis, tumors, and spinal injuries. Symptoms of spinal canal stenosis may include pain, numbness, tingling, weakness, and difficulty walking or maintaining balance. Diagnosis of spinal canal stenosis is usually made through a combination of clinical exams, imaging tests, and nerve function tests. Treatment for spinal canal stenosis may include physical therapy, medication, and surgery in severe cases. The goal of treatment is to relieve pressure on the spinal cord and nerves and manage symptoms. Prognosis for spinal canal stenosis varies depending on the severity of the condition and the effectiveness of treatment. Early detection and prompt treatment are important for improving outcomes and quality of life for affected individuals.
Spinal muscular atrophy (SMA), which is a rare genetic disorder characterized by the loss of nerve cells in the spinal cord and brainstem. This loss of nerve cells leads to muscle weakness and atrophy, particularly in the muscles used for crawling, walking, sitting up, and controlling head movement. SMA is caused by mutations in the SMN1 gene, which is responsible for the production of a protein essential for the survival of motor neurons. The severity of SMA can vary widely, with some forms appearing in infancy and causing severe muscle weakness and respiratory problems, while others appear later in childhood or adulthood and cause milder symptoms. There is currently no cure for SMA, but there are treatments available that can help manage symptoms and improve quality of life, including medication, physical therapy, and assistive devices.
Squamous cell carcinoma (SCC) of the skin, which is a type of skin cancer that arises from the squamous cells, which are the flat cells that make up the outermost layer of the skin. It often develops in areas that are frequently exposed to the sun, such as the face, ears, neck, arms, and hands. SCC can also develop in scars or chronic skin ulcers. The main symptom is the appearance of a red, scaly, and sometimes tender or itchy bump, or a sore that doesn't heal. SCC can spread to nearby tissues or lymph nodes, and in rare cases, it can spread to other parts of the body. Treatment options depend on the size, location, and extent of the tumor, as well as the individual's overall health. Treatment may include surgical removal of the tumor, radiation therapy, or topical chemotherapy. With early detection and treatment, SCC can usually be cured, but advanced cases can be more difficult to treat.
Stroke, which is a serious and often life-threatening condition that occurs when blood flow to the brain is blocked or interrupted, leading to brain damage and a range of neurological deficits.
Sudden cardiac death, which may be determined using a genetic test that assesses an individual's risk of experiencing sudden cardiac death based on specific genetic mutations that are associated with an increased risk.
Syncope, which is a sudden loss of consciousness and muscle tone, often caused by a temporary decrease in blood flow to the brain.
Syringomyelia, which refers to a condition where fluid-filled cysts develop within the spinal cord. Genetic testing can help identify the underlying genetic cause of syringomyelia.
Tachycardia, which is a condition characterized by a rapid heartbeat, usually greater than 100 beats per minute.
Takayasu's arteritis, which is a rare type of vasculitis that affects the large arteries in the body, particularly the aorta and its branches. It is most commonly diagnosed in young women and can cause narrowing, thickening, or inflammation of the arteries, which can restrict blood flow to organs and tissues. Symptoms of Takayasu's arteritis may include fatigue, muscle pain or weakness, fever, headaches, and high blood pressure. Diagnosis of Takayasu's arteritis is typically made through a combination of clinical exams, imaging tests, and blood tests. Treatment for Takayasu's arteritis may involve medications to control inflammation and suppress the immune system, as well as surgery or angioplasty to widen or repair affected arteries. The outlook for individuals with Takayasu's arteritis varies depending on the severity of the condition, the extent of arterial damage, and the effectiveness of treatment. Regular monitoring and management of Takayasu's arteritis are important for improving outcomes and preventing complications such as heart attack or stroke.
T-cell lymphoma, which is a type of cancer that affects the T-cells, which are a type of white blood cell that plays an important role in the immune system.
Temporal arteritis, which is an inflammation of the arteries in the head and neck, causing symptoms such as headaches, jaw pain, and vision loss.
Thoracic aortic aneurysm and dissection, which is a genetic panel that tests for mutations associated with an increased risk of thoracic aortic aneurysm and dissection.
Thoracic aortic aneurysm or dissection, which is a condition in which the wall of the aorta in the chest becomes weakened and bulges outward, putting the person at risk for aortic dissection (a tear in the aorta wall).
Thymoma, which is a rare type of tumor that develops in the thymus gland, which is located in the chest and plays a role in the immune system.
Thyroid cancer pertinent cancer susceptibility, which may be determined using a genetic test that assesses an individual's risk of developing thyroid cancer based on specific genetic mutations that are associated with an increased risk.
Transient ischemic attack, also known as a “mini-stroke”, which is a condition caused by a temporary disruption of blood flow to the brain, leading to temporary neurological symptoms.
Tremor, which is a type of movement disorder characterized by rhythmic, involuntary muscle movements that can affect any part of the body.
Trigeminal neuralgia, which is a chronic pain condition that affects the trigeminal nerve, causing sudden, severe facial pain.
Troyer Syndrome, which is a rare genetic disorder that affects the development of the brain and spinal cord, leading to intellectual disability, muscle weakness, and skeletal abnormalities.
Tuberculosis (TB) susceptibility, which refers to an increased risk of developing tuberculosis, which is an infectious disease caused by the bacterium Mycobacterium tuberculosis.TB most commonly affects the lungs, but it can also affect other parts of the body such as the kidneys, spine, and brain. TB is spread through the air when an infected person coughs or sneezes, and the risk of infection is higher in people with weakened immune systems, such as those with HIV/AIDS, malnutrition, or certain medical conditions. Symptoms of TB may include coughing, fever, weight loss, and night sweats. Treatment for TB involves a combination of antibiotics taken for several months, and it is important to complete the full course of treatment to avoid drug resistance and prevent the spread of the disease. Preventive measures such as vaccination (with Bacillus Calmette-Guérin, or BCG) and infection control practices (such as proper ventilation and use of masks) may also be recommended for people at high risk of TB susceptibility.
Upper gastrointestinal cancer pertinent cancer susceptibility, which may be determined using a genetic test that assesses an individual's risk of developing upper gastrointestinal cancers, such as stomach cancer, based on specific genetic mutations that are associated with an increased risk.
Uveal melanoma, which is a type of cancer that affects the eye.
Vasculitis, which is an inflammation of the blood vessels that can damage organs and tissues throughout the body.
Von Hippel Lindau disease (VHL), which is a rare genetic disorder that can cause tumors to grow in various parts of the body, including the brain, spinal cord, and eyes.
White matter disorders-adult onset, which is a group of rare genetic disorders that affect the myelin sheath, the fatty substance that surrounds and insulates nerve fibers in the brain and spinal cord. Symptoms may include problems with movement, vision, speech, and cognitive function.
White matter disorders-childhood onset, which is a group of genetic disorders that affect the white matter of the brain, leading to neurological symptoms such as developmental delay, seizures, and movement disorders.
Williams Syndrome, which is a rare genetic disorder that affects multiple body systems, causing developmental delay, intellectual disability, distinctive facial features, and cardiovascular abnormalities.
Wilson disease, which is a genetic disorder that affects the body's ability to regulate copper. It can lead to a buildup of copper in the liver, brain, and other organs, causing damage over time.

The genetic-monitoring system 100 disclosed herein may provide valuable insights into individuals' health, risk of disease, and overall genetic makeup with various benefits such as:
Early detection of disease: The genetic-monitoring system 100 disclosed herein may help to identify genetic mutations or variations that are associated with an increased risk of disease. By detecting these mutations early, individuals can take steps to manage their risk, such as getting more frequent medical check-ups or making lifestyle changes to reduce their risk.
Personalized medicine: Genetic monitoring can help to identify genetic variations that affect an individual's response to different medications or treatments. The genetic-monitoring system 100 disclosed herein may enable healthcare/individual providers to develop personalized treatment plans that are tailored to an individual's genetic makeup, increasing the effectiveness and safety of treatment.
Family planning: Genetic monitoring may provide valuable information for family planning, such as identifying the risk of passing on genetic conditions to future children. The genetic-monitoring system 100 disclosed herein may may help individuals and couples make informed decisions about family planning, such as undergoing genetic counseling or pursuing alternative methods of conception.
Research: Genetic monitoring may contribute to ongoing research efforts aimed at understanding the genetic basis of health and disease. By monitoring the genes of large populations over time, researchers can identify patterns and associations that might be difficult to detect in smaller studies.
Personal interest: Some individuals may be interested in monitoring their genes simply out of personal curiosity or interest in their genetic makeup. The genetic-monitoring system 100 disclosed herein may provide insights into their ancestry, genetic predispositions, and other aspects of their genetic heritage.

Therefore, the genetic-monitoring system 100 disclosed herein may provide valuable insights into people's health, risk of disease, and overall genetic makeup. The genetic-monitoring system 100 disclosed herein ensures that genetic monitoring is conducted in an ethical and responsible manner, with appropriate privacy and confidentiality protections in place.

### C. Pharmacogenomics Analysis and Monitoring

In some embodiments, the computer network system 100 may be alternatively or additionally used as a pharmacogenomics-monitoring system for analyzing and and/or monitoring people's potential response to drugs, based on genetic analysis.

As those skilled in the art understand, pharmacogenomics is the study of how an individual's genetic makeup affects their response to drugs. Pharmacogenomics involves the analysis of genetic variations that may affect an individual's ability to metabolize, respond to, or tolerate different medications. Pharmacogenomics generally involves in the following key aspects:
Personalized medicine: Pharmacogenomics enables the development of personalized medicine, where treatments may be tailored to an individual's genetic makeup. This may increase the effectiveness and safety of treatments, while reducing the risk of adverse drug reactions.
Drug discovery: Pharmacogenomics may also be used to identify new drug targets and develop more effective drugs. By identifying genetic variations that are associated with disease susceptibility or drug response, researchers may develop drugs that are targeted to specific genetic variations.
Adverse drug reactions: Pharmacogenomics may help to identify individuals who may be at increased risk of adverse drug reactions. By identifying genetic variations that affect drug metabolism or response, healthcare providers may adjust dosages or prescribe alternative medications to reduce the risk of adverse reactions.
Regulatory considerations: For example, the US Food and Drug Administration (FDA) and other regulatory agencies are increasingly considering pharmacogenomic data in drug development and approval processes. This highlights the importance of incorporating pharmacogenomic data into drug development and clinical decision-making.
Challenges: Pharmacogenomics is a complex and rapidly evolving field with many challenges related to data interpretation, clinical implementation, ethical considerations, and/or the like. It is important to ensure that pharmacogenomic data is used in an ethical and responsible manner, with appropriate privacy and confidentiality protections in place.

Thus, pharmacogenomics may revolutionize drug development and clinical decision-making by enabling the development of personalized medicine and reducing the risk of adverse drug reactions. However, it is important to carefully consider the ethical, legal, and social implications of pharmacogenomics and to ensure that it is used in a responsible and effective manner.

In these embodiments, the pharmacogenomics-monitoring system 100 may be used as a point-of-care (PoC) pharmacogenomics-monitoring system for providing pharmacogenomic analysis to a patient whenever the patient needs.

The pharmacogenomic analysis procedure may be similar to that shown in FIG. 5 except that it is used for determining the patient's potential response to drugs.

For example, once the WGS and/or WES data is obtained, the data may be matched with comprehensive database of pharmaceuticals, and using suitable AI algorithms to automatically provide analysis to the patient. Therefore, if a patient goes to a doctor, and doctor recommended a treatment, the patient may use the pharmacogenomics-monitoring system 100 (or more specifically, use a mobile app thereof, for example) to obtain personalized recommendations and understand in real-time which drugs they may have side effects or which drugs they may respond better.

Similar to the genetic-monitoring system 100 described above, the pharmacogenomics-monitoring system 100 may use suitable AI algorithms to automatically and repeatedly monitored genetic and pharmaceutical research resources to provide patients with updated information and recommendations. The genetic-monitoring system 100 may also help patients to discover new drugs. By using AI, the pharmacogenomics-monitoring system 100 may greatly improve pharmacogenomics by enabling more precise and personalized drug therapy.

In some embodiments, the pharmacogenomics-monitoring system 100 may be linked with telemedicine and relevant healthcare workers to help provide care, and help deliver proper medicines through suitable supply chains.

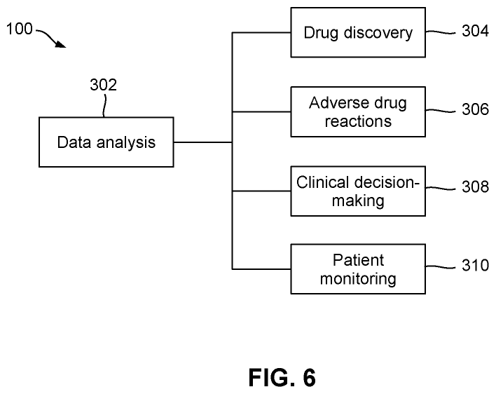

FIG. 6 is a schematic diagram showing the functional structure of the pharmacogenomics-monitoring system 100, according to some embodiments of this disclosure. As shown, the pharmacogenomics-monitoring system 100 may comprise a data analysis module 302 supporting a drug discovery module 304, an adverse drug reactions module 306, a clinical decision-making module 308, and a patient monitoring module 310.

The data analysis module 302 may use suitable AI algorithms to analyze large datasets of genomic and pharmacological data to identify genetic variations that are associated with drug response, which may enable the development of more targeted drug therapies and personalized medicine.

The drug discovery module 304 may use suitable AI algorithms to identify new drug targets and develop more effective drugs by analyzing genomic data and identifying genetic variations that are associated with disease, which may help to identify new therapeutic targets and develop drugs that are more effective and have fewer side effects.

The adverse drug reactions module 306 may use suitable AI algorithms to predict the likelihood of adverse drug reactions based on an individual's genetic makeup and other factors, which may help to identify individuals who are at increased risk of adverse reactions and adjust drug dosages or prescribe alternative medications to reduce the risk of adverse reactions.

The clinical decision-making module 308 may use suitable AI algorithms to assist healthcare providers in making clinical decisions by providing personalized treatment recommendations based on an individual's genetic makeup and other factors, which may help to optimize drug therapy and improve patient outcomes.

The patient monitoring module 310 may use suitable AI algorithms to monitor patients and identify potential adverse reactions or drug interactions based on real-time data, which may help to improve patient safety and reduce the risk of adverse events.

Those skilled in the art will appreciate that, in some embodiments, the methods disclosed herein may be implemented as one or more circuits (such as one or more processors) of a module, a device, an apparatus, a system, and/or the like. In some embodiments, the methods disclosed herein may be implemented as computer-executable instructions stored in one or more non-transitory computer-readable storage media such that, the instructions, when executed, may cause one or more circuits to perform the methods disclosed herein.

Those skilled in the art will appreciate that such various embodiments and/or features thereof may be customized and/or combined as needed or desired. Moreover, although embodiments have been described above with reference to the accompanying drawings, those of skill in the art will appreciate that variations and modifications may be made without departing from the scope thereof as defined by the appended claims.

1.-22. (canceled)

23. A health-analysis method comprising:
obtaining testing data of a patient's sample;
cleaning the testing data by removing data items unrelated to the health analysis;
obtaining statistics of the cleaned testing data; and
obtaining analysis results of one or more genes and/or species of the patient's sample using one or more artificial intelligence (AI) methods based on the obtained statistics of the cleaned testing data and statistics of healthy data.

24. The health-analysis method of claim 23, wherein said obtaining the analysis results of the one or more genes and/or species of the patient's sample comprises:
using a machine learning method for identifying characteristics of the one or more genes, and/or for analyzing genetic data of the testing data and identifying patterns or relationships between different genetic markers;
using a random forest method for building a plurality of decision trees and taking their majority vote for classification;
using a regression method for predicting expression of the one or more genes;
using a clustering method for grouping individuals based on their genetic similarity;
using a maximum relevance minimum redundancy (MRMR) method for selecting a set of features of minimum redundancy and maximum relevance with respect to a target variable;
using a deep learning method for analyzing datasets of the testing data and identifying features and/or patterns;
using a convolutional neural network (CNN) for recognizing patterns in gene expression data of the testing data for identifying one of the one or more genes that is associated with an environmental condition;
using a reinforcement learning method for optimizing genetic datasets by identifying informative genetic features;
using a reinforcement learning method for selecting informative genetic markers for a population while minimizing a number of genetic markers required for predictions;
using a genetic method for optimizing genetic datasets by iteratively testing and refining different combinations of genetic markers;
using a genetic method for identifying an optimal set of genetic markers for predicting a risk of a particular disease or condition; or
a combination thereof.

25. The health-analysis method of claim 23, wherein said cleaning the testing data comprises:
cleaning the testing data using one or more gene panels related to the health analysis.

26. The health-analysis method of claim 23, wherein said obtaining the standard deviations and/or variance of the cleaned testing data comprises:
obtaining standard deviations and/or variance of real read, estimated read, and abundance of the one or more genes from the cleaned testing data.

27. The health-analysis method of claim 23, wherein said obtaining the one or more genes and/or species analysis results comprises:
using the one or more AI models to obtain analysis results regarding similarity between the cleaned testing data and the healthy data and/or ranking of the one or more genes or bacteria based on the obtained statistics.

28. The health-analysis method of claim 23 further comprising:
using the obtained analysis results of the one or more genes and/or species of the patient's sample for determining one or more diseases for the patient, and/or for analyzing and/or monitoring the patient's response to drugs.

29. One or more non-transitory computer-readable storage devices comprising computer-executable instructions, wherein the instructions, when executed, cause a processing structure to perform the method of claim 23.

30. The one or more non-transitory computer-readable storage devices of claim 29, wherein said obtaining the analysis results of the one or more genes and/or species of the patient's sample comprises:
using a machine learning method for identifying characteristics of the one or more genes, and/or for analyzing genetic data of the testing data and identifying patterns or relationships between different genetic markers;
using a random forest method for building a plurality of decision trees and taking their majority vote for classification;
using a regression method for predicting expression of the one or more genes;
using a clustering method for grouping individuals based on their genetic similarity;
using a maximum relevance minimum redundancy (MRMR) method for selecting a set of features of minimum redundancy and maximum relevance with respect to a target variable;
using a deep learning method for analyzing datasets of the testing data and identifying features and/or patterns;
using a convolutional neural network (CNN) for recognizing patterns in gene expression data of the testing data for identifying one of the one or more genes that is associated with an environmental condition;
using a reinforcement learning method for optimizing genetic datasets by identifying informative genetic features;
using a reinforcement learning method for selecting informative genetic markers for a population while minimizing a number of genetic markers required for predictions;
using a genetic method for optimizing genetic datasets by iteratively testing and refining different combinations of genetic markers;
using a genetic method for identifying an optimal set of genetic markers for predicting a risk of a particular disease or condition; or
a combination thereof.

31. The one or more non-transitory computer-readable storage devices of claim 29, wherein said cleaning the testing data comprises:
cleaning the testing data using one or more gene panels related to the health analysis.

32. The one or more non-transitory computer-readable storage devices of claim 29, wherein said obtaining the statistics of the cleaned testing data comprises:
obtaining standard deviations and/or variance of the cleaned testing data.

33. The one or more non-transitory computer-readable storage devices of claim 29, wherein said obtaining the standard deviations and/or variance of the cleaned testing data comprises:
obtaining standard deviations and/or variance of real read, estimated read, and abundance of the one or more genes from the cleaned testing data.

34. The one or more non-transitory computer-readable storage devices of claim 29, wherein said obtaining the one or more genes and/or species analysis results comprises:
using the one or more AI models to obtain analysis results regarding similarity between the cleaned testing data and the healthy data and/or ranking of the one or more genes or bacteria based on the obtained statistics.

35. The one or more non-transitory computer-readable storage devices of claim 29, wherein the method further comprises:
automatically monitoring development in one or more research areas; and
automatically notifying patients and/or healthcare workers regarding changes of functions of the one or more genes and/or changes of ranking of the one or more genes.

36. The one or more non-transitory computer-readable storage devices of claim 29, wherein the method further comprises:
using the obtained analysis results of the one or more genes and/or species of the patient's sample for determining one or more diseases for the patient, and/or for analyzing and/or monitoring the patient's response to drugs.

37. One or more processors functionally coupled to one or more non-transitory computer-readable storage devices, the one or more non-transitory computer-readable storage devices comprising computer-executable instructions, wherein the instructions, when executed, cause the one or more processors to perform the above-described method for performing the method of any one of claim 23.

38. The one or more processors of claim 37, wherein said obtaining the analysis results of the one or more genes and/or species of the patient's sample comprises:
using a machine learning method for identifying characteristics of the one or more genes, and/or for analyzing genetic data of the testing data and identifying patterns or relationships between different genetic markers;
using a random forest method for building a plurality of decision trees and taking their majority vote for classification;
using a regression method for predicting expression of the one or more genes;
using a clustering method for grouping individuals based on their genetic similarity;
using a maximum relevance minimum redundancy (MRMR) method for selecting a set of features of minimum redundancy and maximum relevance with respect to a target variable;
using a deep learning method for analyzing datasets of the testing data and identifying features and/or patterns;
using a convolutional neural network (CNN) for recognizing patterns in gene expression data of the testing data for identifying one of the one or more genes that is associated with an environmental condition;
using a reinforcement learning method for optimizing genetic datasets by identifying informative genetic features;
using a reinforcement learning method for selecting informative genetic markers for a population while minimizing a number of genetic markers required for predictions;
using a genetic method for optimizing genetic datasets by iteratively testing and refining different combinations of genetic markers;
using a genetic method for identifying an optimal set of genetic markers for predicting a risk of a particular disease or condition; or
a combination thereof.

39. The one or more processors of claim 37, wherein said cleaning the testing data comprises:
cleaning the testing data using one or more gene panels related to the health analysis.

40. The one or more processors of claim 37, wherein said obtaining the standard deviations and/or variance of the cleaned testing data comprises:
obtaining standard deviations and/or variance of real read, estimated read, and abundance of the one or more genes from the cleaned testing data.

41. The one or more processors of claim 37, wherein said obtaining the one or more genes and/or species analysis results comprises:
using the one or more AI models to obtain analysis results regarding similarity between the cleaned testing data and the healthy data and/or ranking of the one or more genes or bacteria based on the obtained statistics.

42. The one or more processors of claim 37, wherein the method further comprises:
using the obtained analysis results of the one or more genes and/or species of the patient's sample for determining one or more diseases for the patient, and/or for analyzing and/or monitoring the patient's response to drugs.
